(19) United States
(12) Patent Application Publication
(10) Pub. No.: US 2021/0401332 A1
(43) Pub. Date: Dec. 30, 2021

(54) SELF-MONITORING AND CARE ASSISTANT FOR ACHIEVING GLYCEMIC GOALS

(71) Applicant: MY-VITALITY SÀRL, Founex (CH)

(72) Inventors: Dennis JOHN, Founex (CH); Nilchian MASIH, Saint Sulpice (CH)

(73) Assignee: My-Vitality Sàrl, Founex (CH)

(21) Appl. No.: 17/294,617

(22) PCT Filed: Nov. 6, 2019
(86) PCT No.: PCT/EP2019/080418

§ 371 Date: May 17, 2021

(30) Foreign Application Priority Data
Nov. 15, 2018 (EP) 18206407.1

(51) Int. Cl. A61B 5/145; A61B 5/00; A61B 5/02; A61B 5/021; A61B 5/024; A61B 5/0295

(52) U.S. Cl. CPC A61B 5/14532; A61B 5/7267; A61B 5/02007; A61B 5/0295; A61B 5/02116; A61B 5/02125; A61B 5/02416; A61B 5/746

(57) ABSTRACT

A device, a system for the device and a set of methods used to extract pulse wave features and select an optimal combination of these features for calculating and determining the blood glucose level and discriminating between different sources of blood glucose level changes in a subject, wherein the different blood glucose level changes are selected among the type of nutrients, sport activities, stresses and fatigue or a combination thereof. The system is designed for accurately obtaining, measuring, registering and interpreting the pulse to determine the blood glucose level of a subject. By collecting pulse wave features, selecting those that are most significant and developing algorithms, the device and its method calculates the user's blood glucose levels and discriminates between different sources of blood glucose level changes of the subject.

### FIELD OF THE INVENTION

The invention relates to a self-care device with software and application (app) for healthy individuals and for those who have impaired glucose tolerance or various forms of diabetes. This system is meant to help and encourage users to make the right life style choices for achieving desired glycemic levels. The device and its system extracts and selects a group of identified pulse wave features, which represent an optimal combination of features for calculating and determining levels of glucose in the blood. The designed system provides a more accurate means of obtaining, measuring, registering and interpreting the pulse to determine glucose levels by considering many factors influencing pulse wave form changes.

### BACKGROUND OF THE INVENTION

Glucose, or commonly called sugar, is an important energy source that is needed by all the cells and organs of our bodies. The body maintains blood glucose levels (hereafter “bgl”) within certain limits through various homeostatic mechanisms to ensure the body maintains enough energy without causing large rises in blood sugar levels. In the longer run, poor glucose control leads to both heart and blood vessel disease, kidney failure, nerve damage, eye problems and other complications. Bgl fluctuates considerably during the day especially as per food intake but also per physical activity, levels of sleep, stress, medications and other factors.

Accordingly, there is a large interest in monitoring blood glucose levels (bgl). Many studies have found that the more sugar one consumes the more likely one is to gain weight. Similarly, many studies have found a strong link between poorly controlled blood sugar levels and obesity, Type 2 diabetes and heart disease. Accordingly, a better control of blood sugar levels is of interest in staying healthy whether to prevent or control diabetes or to control or lose weight. To excel in physical activities, we need energy. As energy stores are used up, blood glucose levels fall causing a decline in performance and resulting in fatigue. On the other hand, regular exercise leads to improved insulin controls and thereby improved blood glucose levels. Quality of sleep as well as avoiding excess stress have also a significant influence on blood glucose levels. Accordingly, daily life style choices relating also to sleep quality, stress reduction and physical activity as well as use of medications have an important effect on healthy levels of blood glucose levels whether it is for healthy individuals, those active in sport as well as those with difficulties in maintaining homeostatic levels of glucose.

Currently, monitoring bgl is done primarily by taking regularly blood samples and from the samples measure the glucose concentrations. Numerous attempts have been made to measure bgl through the analysis of the pulse or pulse wave. This includes several efforts at analyzing the pulse wave either in terms of its heart rate or heart rate variability or at looking at the second derivative or “acceleration pulse”. Since blood sampling for bgl is relatively accurate, using non-invasive pulse analysis needs to be accurate otherwise the user is better off taking blood samples despite the inconvenience. Measuring and indicating abnormal glucose levels is critical otherwise hypo- or hyperglycemia can lead to critical health problems. Achieving target glycemic levels using accurate bgl monitoring is also necessary to improve patient outcomes and adapt appropriate life styles including eating habits to those who need to measure regularly bgl. More accurate glucose results may help reduce errors in deciding the amount of carbohydrates intake, insulin dosage or various life style choices. Getting accurate measurements is complicated by the fact that the pulse wave and pulse rate is regularly changing for many reasons other than bgl.

For example, in EP 3 170449 A1, a device and method to detect diabetes is described of taking filtered PPG signals and obtaining the pulse rate peaks and thereby measuring the distance between the consecutive peaks to obtain various features like the mean of the peaks, their standard deviation, and other frequency-based features i.e. heart rates. In addition, the heart rate variability and the PRV (or pulse rate variability) were also calculated using the frequency-domain measures.

While counting heart beats are helpful in indicating blood glucose levels they do not correlate consistently enough with bgl to allow it to be used as a measuring tool. The breakdown and conversion of glucose into cellular energy results in an increased metabolism can manifest itself in the form of increased heart rate. A study by Kennedy and Scholey (“Glucose administration, heart rate and cognitive performance: Effects of increasing mental effort” Psychopharmacology April 2000) demonstrates that people have individualized responses to heightened metabolism, so sugar may not always cause a noticeable change in heart rate for all individuals.

While the heart rates are known to increase or decrease with blood glucose concentrations, this is not enough to accurately measure blood glucose. The heart rate can move disproportionately to bgl especially in situations were the subject has exercised or is under stress. For example, while an increase in bgl may increase the heart rate, increased physical activity will also increase the heart rate but also frequently lead to a decrease in bgl. Mental effort and/or stress can also increase the heart rate independently of bgl.

Studies have shown that heart rate variability is a relatively poor indicator of blood glucose levels. Four hundred and forty-seven participants were classified according to glycemic status in the publication “Influence of blood glucose on heart rate and cardiac autonomic function”, Diabet Med April 2011. It was found that heart rate variability was not associated with glycemic status and capillary glucose. In Applicant's clinical studies identifying correlations between pulse wave features and bgl, heart rate variability was less informative and less indicative of bgl than many other identified pulse wave features.

In EP3289968 A1, pulse rates are used as an indicator of bgl. The patent application also proposes two additional pulse wave features: the augmentation index (AI) and a similar pulse wave feature the stiffness index (SI). The augmentation index (AI) is generally defined as the difference between the first and second peaks of the central arterial waveform, expressed as a percentage of the pulse pressure, and ejection duration time from the foot of the pressure wave to the incisura. AI is a measure of the contribution made by the reflected pressure wave to the ascending aortic pressure waveform. The amplitude and speed of the reflected waves are dependent upon arterial stiffness. The stiffness index is a similar calculation comparing time differences between these two peaks.

Several studies including the results in “The influence of heart rate on augmentation index and central arterial pressure in humans”, Ian Wilkinson and David Webb, The Journal of Physiology, 2000 May 15:525 pp 263-270 demonstrate an inverse, linear relationship between AI and heart rate. This is likely due to alterations in the timing of the reflected pressure wave, produced by changes in the absolute duration of systole. In other words, an increase in heart rate will decrease the absolute duration of systole, effectively shifting the reflected wave into diastole, thereby reducing AI. Accordingly, these identified features are of limited use in identifying additional correlations with bgl beyond what is known regarding heart rate.

Other studies demonstrate little correlation between arterial stiffness and bgl. In “Effects of glucose control on arterial stiffness in patients with Type 2 diabetes mellitus and hypertension: An observational study” Sangah Chang and Jungmin Lee, Journal of International medical Research, 2018 Vol 46 (284-292), it was concluded that short-term glycemic control did not influence the arterial stiffness in patients with type 2 diabetes mellitus.

A study was performed on several subjects for four days where the subjects ate rice and stew (including red meat and vegetables) to investigate the relationship between glucose level and (AI/SI). SI's and AI's are measured 5 min after intake as are the glucose levels using a glucose measuring device where a blood sample is taken with each test. The same process is done for another four days, while the subjects eat 400 gr banana. In both cases glucose levels increase, while AI decreases after eating rice and stew and AI increases after eating banana and there were no significant changes in SI values.

In another study, the subjects were asked to drink one bottle of 500 ml Fanta and take the glucose blood test as well as monitor the subject's pulse waves like the prior described study also for four days. After the glucose drink, blood glucose level and AI both increases. However, the AI levels increased at significantly lower rates. SI remained relatively constant. After two hours, even though the blood glucose levels returned to the same value before the drink the AI and the SI values stayed high.

In EP3269305 A1, the document discusses the use of an “accelerated pulse wave” or commonly referred to as the second derivative. Changes in the inflection points of the pulse wave are better visualized using the second derivative allowing a more accurate calculation of the peaks and notches as per changes from the baseline. The AI and SI are often calculated from the acceleration pulse wave. The heights of these main inflection points are used for analysis.

It was stated that acceleration pulse wave is correlated with the glucose level, which is not the case in general for example after drinking a glucose drink. Applicants in the stew and rice, banana and Fanta studies, found little correlation between bgl and accelerated pulse wave. There was also little to no correlation between the different food samples taken in these studies and the ratio of the first and the second peak of the acceleration pulse wave.

There is high complexity of measuring blood glucose level without taking any blood (non-invasive). It has been observed that AI increases after eating banana and AI decreases after eating carbohydrates and fats while in the both cases glucose level increases.

After glucose drink, blood glucose level and AI both increases. However, after two hours, even though the blood glucose level return back to the same value before the drink, the AI and the SI values stay high.

Indeed, these results show that AI and SI aren't correlated with the glucose level.

In addition, acceleration pulse wave (e.g. ration of the first and the second peak amplitudes) isn't correlated with the blood glucose level neither.

It has been observed that blood glucose level not only changes by eating and drinking, but it also varies after sport activities, fatigue and stress. Pulse wave forms are constantly changing. There are many factors that can change the form of the pulse wave. Exercise, breathing rate, movement, metabolism, stress, different types and quantities of food consumption are examples of this. This makes identifying pulse wave features that specifically change or are specifically correlated to blood glucose level changes especially challenging.

As discussed there is not any linear relationship between glucose level and AI, SI, HR, HRV, and acceleration pulse wave, in general. The relation between glucose level and AI/SI depends on whether the subject is healthy, pre-diabetic and/or diabetic.

It can be the reason why the designed devices of the prior art have not been put into practice. It is thus highly helpful to design an electronic device that can measure blood glucose level in a non-invasive manner.

In addition, it is difficult to non-invasively measure and to determine levels of glucose in the blood because of a lack of standards needed to make and verify these measurements. There are no single sets of biomarkers or other standards since there are different causes of bgl and because it manifests itself in different ways.

### BRIEF DESCRIPTION OF THE INVENTION

The electronic pulse wave device of the invention first determines whether the subject is diabetic, pre-diabetic or healthy, then determines the source of blood glucose level changes selected among the type of nutrients, type of sport activities, and type of stresses and fatigue. It then estimates the blood glucose numerical range based on the model corresponding to the determined source of blood glucose level change. It then applies the developed recurrent i.e. neural network to analyze the time series of the blood pulse wave accordingly and estimates blood glucose level with higher precision.

The circulatory system allows blood glucose levels to be regulated. After one eats, the digestive system breaks down carbohydrates and turns them into glucose. As one's sugar levels rise, the pancreases releases insulin, which helps regulate glucose levels. Inside your cells, the glucose is burned to produce heat and adenosine triphosphate (ATP), a molecule that stores and releases energy as required by the cell. Glucose is converted to energy with oxygen in the mitochondria. This conversion yields energy plus water and carbon dioxide. Glucose is also converted to energy in muscle cells. Muscle cells have mitochondria, so they can process glucose with oxygen. But if the level of oxygen in the muscle cell falls low, the cells change glucose into energy without it.

Many of these changes are reflected in physiological changes in the blood circulatory system. One of know mechanisms is the narrowing of the blood vessels with higher glucose levels. This response, in turn, influences blood flow, blood pressure and the general pulse wave form. Other influences on the blood circulatory system include: metabolism, changes in heart rate, changes in breathing rate and changes in hormone levels especially insulin.

This invention consists of establishing correlations between pulse wave form changes and different levels of blood glucose through these physiological changes on the blood circulatory system. This approach relies on analyzing the physiological characteristics of the cardiovascular system as indicated by variations observed on the pulse wave form.

Bgl can change based on the quantities and types of foods eaten, sleeping patterns, physical activity, stress and other daily influencing factors. Knowing these and how they inter relate with each other can improve the determination of bgl. Assembling the data from these other factors and related indications into one system and device will also help the user make and improve on their life style choices to better manage bgl.

One of the objects of the present invention is to provide a statistical and analytic non-invasive method for interpreting a set of pulse wave recordation of a subject for quantifying the blood glucose level and/or discriminating between different sources of blood glucose level changes selected among the type of nutrients, type of sport activities, type of stresses and fatigue or a combination thereof, said method comprising the steps of:
extracting and selecting from said set of pulse wave recordation each single pulse wave and its first and second derivation so as to obtain a first set of features providing information data consisting in the time, amplitude, area, ratios, heart rate and breathing rate;
characterized in that, the method is performing a statistical analysis on the collected information data from the pulse wave and/or on said first set of features obtained from at least two single pulse waves to arrive at a second set of features providing additional information data consisting in the mean, variation around the mean, randomness and/or time series analysis between said first set of features of the at least two single pulse waves; and wherein the method is combining said first and second set of features and applying means configured in a software to analyze, determine and display the results of the blood glucose level and/or of the discrimination between different sources of blood glucose level changes of said subject.

According to another exemplary embodiment, a pulse wave device for determining and quantifying the level of bgl may be applied on a pulse-taking location on the body of said subject. However, this invention is not confined to physically getting pulse waves on parts of the body through ppg. Any means of getting a pulse wave is accepted. This can include for instance using a camera on a smart phone or otherwise and capturing the pulse wave through camera generated images.

In particular, the invention provides a pulse wave device for quantifying the blood glucose level in a subject and/or for discriminating between different sources of blood glucose level changes, wherein blood glucose level changes are selected among the type of nutrients, type of sport activities, type of stresses and/or fatigue or a combination thereof, said pulse wave device being applied on a pulse-taking location on the body of said subject; said pulse wave device comprising:
a sensor module (1) for collecting information data from the pulse wave, a memory module (4) for storing the pulse wave information data on the pulse wave device, a display module (3) for displaying the results of the blood glucose level and/or the discrimination between said different sources of blood glucose level changes and a processor module (2) comprising:
means of extracting and selecting from each single pulse wave and from its first and second derivation a first set of features providing information data consisting in the time, amplitude, area, ratios, heart rate and breathing rate;
characterized in that, said processor module (2) is configured to perform a statistical analysis on the collected information data from the pulse wave and/or on said first set of features obtained from at least two single pulse waves to arrive at a second set of features providing additional information data consisting in the mean, variation around the mean, randomness and/or time series analysis between said first set of features of the at least two single pulse waves; and wherein, said processor module (2) further comprises means for combining said first and second set of features and means to analyze and display the results of the blood glucose level and/or the discrimination between said different sources of blood glucose level changes of said subject.

The device is also intended to assist the user better control through bgl by providing helpful related information. This includes but not excluded to: sleep and sleep related indications, physical activity levels, a log where the user can input regularly food intake information and other related information related to controlling bgl and stress and fatigue indications.

This invention also includes at least two methodologies for obtaining an optimal group of pulse wave features for determining bgl. In the first methodology described, pulse wave features are pre-selected using a set of mathematical methodologies similar to machine learning to obtain optimal correlations with bgl. From these described mathematical steps, a group of pulse wave features are found to be informative in measuring bgl. A set of calculations are thereafter described to group these identified features into an optimal group of features for measuring bgl. A second methodology is described and used to further refine and chose a group of pulse wave features that correlate optimally for measuring bgl. Here deep learning as a mathematical methodology is described for obtaining further an optimal group of pulse wave features for determining bgl. Deep learning is necessary as a means also of discriminating bgl under different scenarios and conditions. To obtain more precise correlations deep learning considers other factors such as stress, physical activity, sleep and food intake to obtain a dynamic model —without a preselection of pulse wave features or conditions - which can adjust to the changing circumstances that affect bgl.

Other objects and advantages of the invention will become apparent to those skilled in the art from a review of the ensuing detailed description, which proceeds with reference to the following illustrative drawings, and the attendant claims.

### BRIEF DESCRIPTION OF THE FIGURES

Advantages of embodiments of the present invention will be apparent from the following detailed description of the exemplary embodiments thereof, which description should be considered in conjunction with the accompanying figures in which like numerals indicate like elements, in which:

FIG. 1 is an exemplary embodiment of a circuit diagram showing an example of some of the main components in a circuit configuration of a pulse wave extraction and recording device. Specifically, FIG. 1 depicts: a sensor module (1) for collecting information data from the pulse wave, a memory module (4) for storing the pulse wave information data on the pulse wave device, a display module (3) for displaying the bgl and a processor module (2) comprising a software.

FIG. 2 is an exemplary embodiment of a visual image of a battery, which may be provided as a way of depicting in an easily understandable bgl.

FIG. 3 is an exemplary embodiment of a diagram in a set of modules which may show a method for collecting pulse waves for a period of time and identifying a set of individual pulse waves of quality.

FIG. 4 is an exemplary embodiment of a diagram of a single pulse wave which may depict a systolic peak, a diastolic peak, a dicrotic notch, the first and the last points corresponding to the half-height of the systolic peak with their times, and amplitudes of the single pulse wave.

FIG. 5 is an exemplary embodiment of a diagram of a pulse wave whose diastolic peak is challenging to identify. It also depicts its first and second derivative curves. The diastolic peak and the dicrotic notch is identified using the second derivative of the pulse wave.

FIG. 6 is an exemplary embodiment of a diagram in a set of modules which may show the method by which the first set of features of pulse wave (characteristic features) are obtained from the pulse wave timeline and its seven points: systolic peak, diastolic peak, dicrotic notch, starting and ending point, and the first and the second points corresponding to the half-height of the systolic peak. Original features may be obtained from the pulse wave by applying the calculations of time, amplitude, area, and ratios.

FIG. 7 is an exemplary embodiment of a diagram depicting a final step in the illustrated method of FIG. 6. As a final step in this illustrated method, the second set of features may be obtained by calculating, for each feature in the first set of features, its respective mean, variance, skewness and entropy.

FIG. 8 is an exemplary illustration of the correlation between two features. The darker images on the grayscale presents those combinations of features that are independent or complementary from each other. Conversely, lighter images depict higher levels of inter-relationship.

FIG. 9 is an exemplary embodiment of a diagram showing a much-simplified illustration of the methodology used to obtain an optimal set or group of features as an indication of levels of bgl. The anova math method including the F-test technique may be used to identify the pulse wave features most useful to determine bgl. The method purposes to narrow down the number of features to around 70. From these 70 features, various sparse math techniques are used to identify sub-sets or groups of features best permit differentiation. Upon the identification of around 20 sets or combinations of features that show correlation with various aspects of bgl, the features in each group are replaced one by one with the other features to continue to get the best sub-sets of features. By repeating these steps a few times such as five times, a best group or optimal sub-sets or combination of features are identified.

FIG. 10 is a diagram showing the steps taken in this methodology to obtain the blood glucose levels starting with the pulse wave collection.

FIG. 11 is graph showing relationship between the AI level and bgl over time points.

FIG. 12 represents 4 time graphs depicting bgl levels as it relates to AI and e SI (comparing the effect of eating before and afterwards bananas and rice with stew).

FIG. 13 represents 4 time graphs depicting bgl levels as it relates to AI and SI (comparing the effect of eating bananas and drinking Fanta before and afterwards).

FIG. 14 second derivative wave depicting the ratio of accelerated pulse wave over time of Fanta study and rice with stew study.

FIG. 15 Scatter plot showing relationship between AI and bgl and SI and bgl.

FIG. 16 Scatter plot showing relationship between acceleration wave and bgl.

FIG. 17 illustrates the use of RNN for decision making model.

FIG. 18 Plot depicting the skewness of ratio of systolic area and diastolic area by time: baseline (before bread or stead or glucose drink), after bread or stead and after glucose drink.

FIG. 19 Plot depicting the time difference between the ending point and the systolic by time: baseline (before bread or stead or glucose drink), after bread or stead and after glucose drink.

FIG. 20 Plot depicting the ratio of diastolic area and the amplitude of diastolic peak by glucose value ranges.

FIG. 21 Plot depicting the skewness of the ratio of the amplitude of systolic by the time of systolic by time: before, after, one hour after, and two hours after glucose drink.

### DETAILED DESCRIPTION OF THE INVENTION

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

In the case of conflict, the present specification, including definitions, will control.

Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise” is generally used in the sense of include, that is to say permitting the presence of one or more features or components.

Some embodiments may be described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequences of actions described herein can be considered to be embodied entirely within any form of computer readable storage medium having stored therein a corresponding set of computer instructions that upon execution would cause an associated processor to perform the functionality described herein. Thus, the various aspects of the invention may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

As used in the specification and claims, the singular forms “a”, “an” and “the” include plural references unless the context clearly dictates otherwise.

The presence of broadening words and phrases such as “one or more,” “at least,” “but not limited to” or other like phrases in some instances shall not be read to mean that the narrower case is intended or required in instances where such broadening phrases may be absent.

As used herein the terms “subject” or “patient” or “individual” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder. However, in other embodiments, the subject can be a normal subject. The term does not denote a particular age or sex. Thus, adult and newborn subjects, whether male or female, are intended to be covered.

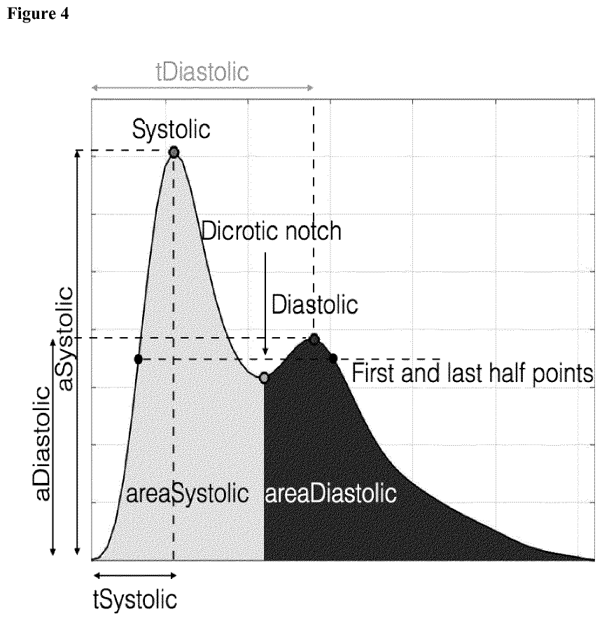

A “pulse wave” (PW) is the progressive increase of pressure radiating through the arteries that occurs with each contraction of the left ventricle of the heart. In other words, a pulse wave (PW) is a measure of the change in the volume of arterial blood with each pulse beat. Specifically, the arterial pulse waveform is a contour wave generated by the heart when it contracts, and it travels along the arterial walls of the arterial tree. Generally, there are 2 main components of this wave: a forward moving wave and a reflected wave. The forward wave is generated when the heart (ventricles) contracts during systole. This wave travels down the large aorta from the heart and gets reflected at the bifurcation or the “cross-road” of the aorta into 2 iliac vessels. In a normal healthy person, the reflected wave usually returns in the diastolic phase, after the closure of the aorta valves. The returned wave which gives a notch pushes the blood through the coronaries. As shown in FIG. 4, seven main timeline points can be used to obtain pulse wave features: (1) starting point, (2) first point corresponding to the half-height of the systolic peak (3) Systolic peak (4) Dicrotic notch (5) Diastolic peak and (6) last point corresponding to the half-height of the systolic peak and (7) ending point.

As used herein “blood glucose level” or “bgl” is the amount of glucose in the blood. Glucose is a sugar that comes from the foods we eat, and it's also formed and stored inside the body. It's the main source of energy for the cells of our body, and it's carried to each cell through the bloodstream. Bgl monitoring is measuring bgl for assessing or controlling these levels and includes determining the presence or likelihood of diabetes. This includes not only the presence of diabetes but also its progressions, changes in levels of, the likelihood of, the probability of having, not having or developing or not developing diabetes. Diabetes includes Type I, Type II, pre-diabetes, hyperglycemia impaired fasting glucose, impaired glucose tolerance.

As used herein, photoplethysmography (PPG) is an optical measurement technique that can be used to detect blood volume changes in tissue. PPG refers to a sensing technique that exploits the change of light absorption that is observed in human's tissue due to changes in blood volume. Each time a heart beat occurs, a pressure wave travels along the arteries thereby increasing the diameter of the artery segment measured. By analyzing the absorption of light one obtains these blood volume changes.

“Fatigue” may also be referred to in such terms as exhaustion, weakness, lethargy, tiredness, describe a general physical and/or mental state of being or feeling weak, lacking energy, lacking vitality, zeal or zest, lacking strength, apathy, feeling “often tired”, etc. Fatigue is one of the most commonly encountered complaints in medical practice. In Western medicine, it is characterized by feelings of low levels of energy, a lessened capacity or motivation to work or be active, and often accompanied by sleepiness and weakness. In Chinese Traditional Medicine (TCM) and other oriental medicine, they refer to this condition as lacking Qi or lacking energy. Qi is considered generally your life force or vital energy, which circulates in and around all of us. This Qi can stagnate or be blocked and a significant part of TCM involves “unblocking” or releasing this Qi.

Physical and mental fatigue and lack of sleep also referred herein as fatigue related to sleep troubles are three main sources of fatigue. They can often exist together even though they arise from different causes. Stress, anxiety, worry, depression or emotional grief can result in physical feelings of exhaustion even though the main source of fatigue is not from physical exertion. Similarly, extended periods of access physical activity can result in feelings of stress and anxiety. The result is that an individual will have a general feeling of tiredness of a more chronic nature than a short term feeling of exhaustion, such as might be caused by, for example, a lack of sleep or a lot of physical exercise. With a general feeling that one has a lack of energy reserves or that the “battery is low”, such tiredness can manifest itself in such emotional states as lethargy, lack of ambition or even have a direct effect physically such as a weakness of the immune system, making one more prone to colds/flues or other ailments.

Within the more general area of fatigue, there are more specific sources, indicators or factors of fatigue where there is also need for measurement and monitoring. Those “different sources of fatigue” or “fatigue related indicators” or factors are selected among physical fatigue, mental fatigue, fatigue related to lack of oxygen, fatigue related to sleep troubles, fatigue related to stress or a combination thereof. In particular, physical fatigue may include overload, performance, VO2 max, first and second ventilatory threshold, discrimination or differentiation between overreach and non-overreach in sports activity and differentiation between a well-recovered state and a non-well-recovered state in sports activity. On the other hand, fatigue related to sleep troubles may include somnolence or drowsiness, sleep deprivation, lack of sleep efficiency, lack of deep sleep lack of light sleep and/or lack of REM (Rapid Eye Movement).

“Heart Rate Variability” (HRV) is the physiological phenomenon of variation in the time interval between heartbeats. It is measured by the variation in the beat-to-beat interval. Other terms used include: “cycle length variability”, “RR variability” (where R is a point corresponding to the peak of the QRS complex of the ECG wave; and RR is the interval between successive Rs), and “heart period variability”.

“Blood pressure” (BP) is the pressure of circulating blood on the walls of blood vessels. When used without further specification, blood pressure usually refers to the pressure in large arteries of the systemic circulation. Normal fluctuation in blood pressure or “blood pressure change” is adaptive and necessary. Studies have shown, for example, that a lack of sleep can limit the body's ability to regulate stress hormones, leading to higher blood pressure.

“Stress” is a physical, mental, or emotional factor that causes bodily or mental tension. Stresses can be external (from the environment, psychological, or social situations) or internal (illness, or from a medical procedure). Stress can initiate the “fight or flight” response, a complex reaction of neurologic and endocrinologic systems. Several of the many physiological changes from stress include: acceleration of heart and lung action; constriction of blood vessels in many parts of the body; liberation of nutrients (particularly fat and glucose) for muscular action; dilation of blood vessels for muscles.

The term “video plethysmography” refers to obtaining recordings of a subject's face, hands, fingers or any other body location where it is possible to extract a pulsatile signal or PPG signal, which is caused by arterial pulsations in the body flow. These color variations in the skin's surface are obtained using a photo detector pointed towards a subject's skin surface and recording the area and thereafter extracting the pulse wave signals from the color variations. Cameras integrated in mobile phones or smart phones permit and easier integration of recordings with an app or apps along with the related software needed to process the data and display the results such as bgl on the smart phone screen.

The “accelerated pulse wave” refers to the “second derivative” pulse wave. The quality of the PPG signal can vary based on motion, light and other artifacts. The first and second derivative of the PPG signal is useful for facilitating the interpretation of the original PPG signals. These derivatives allow more accurate recognition of the inflection points. The second derivative is more commonly used than the first derivative. It is also called the acceleration pulse wave as it is an indication of the acceleration of the blood. The changes in the inflection points of the pulse wave are better visualized thereby allowing a more accurate calculation of the peaks and notches as per changes from the baseline. The AI and SI are often calculated from the acceleration pulse wave. The heights of these main inflection points are used for analysis.

An “app” is an abbreviated form of the word “application.” An application is a software program that's designed to perform a specific function directly for the user or, in some cases, for another application program especially as downloaded by a user to a mobile device.

In the present invention, the term “discrimination” or “discriminating” means making a distinction between different sources of bgl and the health status of the subject as it relates to diabetes.

It is the ability to recognize or draw fine distinctions between different sources of bgl in a subject.

“Metabolism” all the chemical processes in the body, especially those that cause food to be used for energy and growth. Metabolism is the sum total of the physical and chemical processes that occur in the body after eating that breaks down the food into digestive particles and converts the food intake into energy and eliminates the waste materials.

During and after eating there is a greater demand on the body such as supplying glucose for working muscles and the other functions of metabolism. In order to isolate the effects of different foods on bgl it is necessary to identify the effects of the metabolism functions on the heart rate, heart function and the pulse wave. By identifying the changes in pulse wave features that are common with eating, it is possible to examine those pulse wave features that are correlated to bgl without the distortions or other changes on pulse wave features due to the other metabolic functions. The identification of the pulse wave features correlated with bgl is thereby isolated, neutralized or indexed so as to make them the same regardless of the type of foods consumed. A drink requires less digestive function that eating stew and rice. Yet, a drink can have as much or possible more influence on bgl than the rice and stew. By identifying the common pulse wave features that occur from eating it is then possible to group the results or compare the results of a drink with rice and stew even though the body uses more energy to digest these two different foodstuffs.

The terms “sub-set of features” represents an exemplary embodiment of a combination of features (resulting from the combination of the first set of features step a) and the second set of features of step b)) which may allow the determination of more accurate or precise levels of bgl in a subject. In an exemplary embodiment, an optimal set of features corresponding to specific bgl related indicators may be obtained, whereas in other exemplary embodiments there may be other combinations that work but are less effective.

In mathematics or statistics, a “combination” is a way of selecting items from a collection, such that (unlike permutations) the order of selection does not matter. A combination is a selection of all or part of a set of objects or features, without regard to the order in which objects or features are selected.

The “mean” is the average of the numbers, a calculated “central” value of a set of numbers. The “first and the last half points” are the first and the last points on the curve of the pulse wave having values equal to half of the values of the systolic peak amplitude, respectively.

As used in the present disclosure, “variation around the mean” is meant as including skewness, variance, entropy and standard deviation as defined below.

In probability theory and statistics, “skewness” is a measure of the asymmetry of the probability distribution of a real-valued random variable about its mean. The skewness value can be positive or negative, or even undefined.

“Variance” is a measurement of the spread between numbers in a data set. The variance measures how far each number in the set is from the mean. Variance is calculated by taking the differences between each number in the set and the mean, squaring the differences (to make them positive) and dividing the sum of the squares by the number of values in the set.

“Entropy” is a measure of randomness. Entropy is used to help model and represent the degree of uncertainty.

The “standard deviation” is a measure of the spread of scores within a set of data. By “derivatives of waveforms” it is meant that the first derivative is the velocity of the curve and the second derivative shows the acceleration or how fast the velocity of the curve changes.

The “ratio” means the division of two or more features or any function of features, and also includes the subtraction of at least two features and any function of features.

“Augmentation index” or “AI” is a ratio consisting of dividing from the blood pulse wave the height or amplitude of the systolic peak from the height or amplitude of the diastolic peak. A variation of this ratio is to subtract the height of the dicrotic notch from these two described peaks.

“Stiffness Index” or “SI” is similar to the “Augmentation Index” but instead of dividing the amplitudes of the systolic and diastolic peaks, the time differences between these two peaks are compared. A variation on this is to calculate the pulse transit time between and ECG and a PPG recording and comparing them or comparing these points with different pulse waves.

The “power spectrum” of a signal describes the distribution of power into frequency components composing that signal.

“Machine learning” is the science of getting computers to learn and act like humans do, and improve their learning over time in autonomous fashion, by feeding them data and information in the form of observations and real-world interactions. The fundamental goal of machine learning algorithms is to generalize beyond the training samples i.e. successfully interpret data that it has never ‘seen’ before.

“Deep learning” as used in the present invention is a collection of algorithms used in machine learning, used to model high-level abstractions in data through the use of model architectures, which are composed of multiple nonlinear transformations. It is part of a broad family of methods used for machine learning that are based on learning representations of data. Deep learning is a specific approach used for building and training neural networks, which are considered highly promising decision-making nodes. An algorithm is considered deep if the input data is passed through a series of nonlinearities or nonlinear transformations before it becomes output. In contrast, most modern machine learning algorithms are considered “shallow” because the input can only go only a few levels of subroutine calling.

Deep learning removes the manual identification of features in data and, instead, relies on whatever training process it has to discover the useful patterns in the input examples. This makes training the neural network easier and faster, and it can yield better results as it applied to measuring bgl.

Within deep learning, this invention uses much of but not exclusively to deep learning methods: Recurrent neural network and convolutional neural networks.

“Recurrent neural network” or “RNNs” are a recurrent neural network is a class of artificial neural network where connections between nodes form a directed graph along a sequence. This allows it to exhibit temporal dynamic behavior for a time sequence. The use of recurrent neural networks as a methodology in obtaining bgl is illustrated in FIG. 17. They are especially powerful in use cases in which context is critical to predicting an outcome and are distinct from other types of artificial neural networks because they use feedback loops to process a sequence of data that informs the final output, which can also be as a sequence of data. These feedback loops allow information to persist.

In some cases, artificial neural networks process information in a single direction from input to output. These “feedforward” neural networks include convolutional neural networks that underpin image recognition systems. RNNs, on the other hand, can be layered to process information in two directions.

A “convolutional neural network” (CNN) is a type of artificial neural network used primarily in image recognition and processing that is specifically designed to process pixel data. CNNs are powerful image processing that use deep learning to perform both generative and descriptive tasks, often using machine vison that includes image and video recognition, along with recommender systems and natural language processing. This neural network has their “neurons” arranged in such a way as to cover the entire visual field avoiding the piecemeal image processing problem of traditional neural networks.

The layers of a CNN consist of an input layer, an output layer and a hidden layer that includes multiple convolutional layers, pooling layers, fully connected layers and normalization layers. The removal of limitations and increase in efficiency for image processing results in a system that is far more effective, simpler to trains limited for image processing and natural language processing.

“Time series analysis” comprises methods for analyzing time series data in order to extract meaningful statistics and other characteristics of the data.

“Time series data” is a set of observations on the values that a variable takes at different times.

“ANN” refers to an artificial neural network is a network of simple elements called artificial neurons, which receive input, change their internal state (activation) according to that input, and produce output depending on the input and activation.

In an exemplary embodiment, the invention provides a statistical and analytic non-invasive method for interpreting a set of pulse wave recordation of a subject for quantifying the blood glucose level and/or discriminating between different sources of blood glucose level changes selected among the type of nutrients, type of sport activities, type of stresses and fatigue or a combination thereof, said method comprising the steps of:
extracting and selecting from said set of pulse wave recordation each single pulse wave and its first and second derivation so as to obtain a first set of features providing information data consisting in the time, amplitude, area, ratios, heart rate and breathing rate;
characterized in that, the method is performing a statistical analysis on the collected information data from the pulse wave and/or on said first set of features obtained from at least two single pulse waves to arrive at a second set of features providing additional information data consisting in the mean, variation around the mean, randomness and/or time series analysis between said first set of features of the at least two single pulse waves; and wherein the method is combining said first and second set of features and applying means configured in a software to analyze, determine and display the results of the blood glucose level and/or of the discrimination between different sources of blood glucose level changes of said subject.

Preferably, time series analysis are performed by ANN, RNN, DL or CNN techniques.

According to an embodiment of the invention, the statistical and analytic non-invasive method is further adapted to identify diabetic or pre-diabetic subjects from healthy subjects and wherein diabetes or pre-diabetes comprises Type I diabetes, Type II diabetes, hyperglycemia impaired fasting glucose and impaired glucose tolerance.

The software calculates the pre-selected combination of features after the preprocessing step involving the selection of convenient or good pulse waves and then applies it to the model programmed in the software to determine bgl.

The “pre-processing step” is the software development necessary prior to having a software program ready and in completed form to process collected pulse waves and apply selected features and algorithms to the data to estimate bgl. The algorithms developed upon the selection of optimal pulse wave features are integrated into software so that the software can then go through the necessary calculations and display the results in a set of visuals as shown by way of example in FIG. 2. The pre-processing or software development includes programming that can take into consideration in the calculation various specific attributes of each individual such as age, gender, health conditions and other factors that might have an effect on the overall quantification of bgl.

The invention also includes the option to combine bgl data acquired from invasive or semi-invasive means such as taking blood samples with the methodology described in this invention.

This may be useful to help calibrate the device from time to time to more accurately estimate bgl using the non-invasive methodology. Accuracy could be improved upon by using invasive acquired data to check or correct or adjust non-invasively calculated bgl. This could be especially helpful in more critical situations or where the user needs as accurate an estimate as possible. It might also be acquired under certain conditions for regulatory compliance or as a means for the user to double check or confirm the non-invasive estimated bgl. The user will feel more comfortable using non-invasive estimates if assured that they track well with blood tested or other more standard glucose monitoring technique. Combining the data sets also enables the methodology to learn from the invasive data as described in this invention and through the learning process to improve the accuracy of current and future calculations of bgl.

In the frame of the invention, “calibration” is the fact of correlating the estimates of bgl from the invention's methodology with data acquired using standard bgl measurements such as blood samples or semi-invasive sampling through continuously glucose monitoring techniques or otherwise in order to check the methodology and device's accuracy.

According to an exemplary embodiment, pulse waves may have been collected and recorded beforehand, namely before carrying out the steps of the method. It is therefore noted that, according to such an embodiment, no diagnostic method involving the presence of a medical doctor or the subject (patient) is performed by performing all the steps of the method.

According to a preferred embodiment, the first set of features may be determined by measuring the entire pulse wave timeline, or by identifying a set of pulse wave points. In an embodiment, points may be selected from the following points: the systolic peak, diastolic peak, dicrotic notch, the first and last points corresponding to the half-height of the systolic peak, and the starting and ending points of said single pulse wave.

Preferably, the ratios in said first set of features may include the following:
A ratio of an amplitude of a systolic peak and an amplitude of a diastolic peak;
A ratio of the amplitude of the systolic peak and an amplitude of a dicrotic notch;
A ratio of the amplitude of the dicrotic notch and the amplitude of the diastolic peak;
A ratio of a time value of the systolic peak and a time value of the diastolic peak;
A ratio of the time value of the systolic peak and a time value of the dicrotic notch;
A ratio of the time value of the dicrotic notch and the time value of the diastolic peak;
A time difference between the time value of the systolic peak and the time value of the diastolic peak;
A time difference between the time value of the systolic peak and the time value of the dicrotic notch;
A time difference between the time value of the dicrotic notch and the time value of the diastolic peak;
A local cardiac output corresponding to a ratio of an area under the curve to a time difference between a starting time and an ending time;
A ratio of the area under the curve between the starting point and the systolic peak to the amplitude of the systolic peak;
A local systolic cardiac output corresponding to a ratio of an area under the curve between the starting point and the dicrotic notch to the time value of the dicrotic notch;
A ratio of an area under the curve between the starting point and the dicrotic notch to the amplitude of the systolic peak;
A local diastolic cardiac output corresponding to a ratio of an area under the curve between the dicrotic notch and the ending point to the time difference between the time value of the dicrotic notch and the time value of the ending point;
A ratio of an area under the curve between the dicrotic notch and the ending point to the amplitude of the diastolic peak;
A pulse width at ten, thirty, fifty, seventy, or ninety percent corresponding to a time difference between the first and the last points corresponding ten, thirty, fifty, seventy, or ninety percent of the systolic peak, respectively;
A time difference between the first point corresponding to ten, thirty, fifty, seventy, or ninety percent of the systolic peak and the systolic time;
A time difference between the systolic peak and the last point corresponding to ten, thirty, fifty, seventy, or ninety percent of the systolic peak;
A pulse interval corresponding to the time difference between the ending and starting time;
A slope of the systolic peak corresponding to the ratio of the amplitude of the systolic peak by the time value of the systolic peak;
A slope of the diastolic peak corresponding to the ratio of the amplitude of the diastolic peak by the time difference between the ending point and the diastolic peak;
A diastolic decay corresponding to a logarithm of the slope of the diastolic peak;
An inflection point area ratio corresponding to the ratio of the area under the curve between the dicrotic notch and the ending point divided by the area under the curve between the starting point and the dicrotic notch;
An augmentation index, corresponding to the ratio of the amplitude of the systolic peak divided by the amplitude of the diastolic peak;
the ratio of the local diastolic cardiac output by the local systolic cardiac output, or the inverses thereof;
A pulse mean corresponding to the mean of the pulse curve;
A pulse standard deviation corresponding to the standard deviation of the pulse curve;
A pulse median corresponding to the median of the pulse curve;
A ratio of the local systolic cardiac output and the local diastolic cardiac output;
A ratio of the amplitude of the systolic peak minus the amplitude of the dicrotic notch divided by the amplitude of the diastolic peak minus the amplitude of the dicrotic notch;
A ratio of the area under the curve between the systolic peak and the dicrotic notch to the time difference between the time of the systolic peak and the time of the dicrotic notch;
A ratio of the area under the curve between the systolic peak and the dicrotic notch to the amplitude of the systolic peak.

The variation around the mean in said second set of features may include skewness, variance and standard deviation.

Preferably, the randomness in said second set of features may include entropy.

According to a preferred embodiment, the means configured to analyze, determine and display results of bgl of said subject may include a software configured to calculate the result of the bgl in a predetermined and recommended manner.

According to an exemplary embodiment, the software is configured to calculate a pre-selected combination of said first and second set of features after a preprocessing step involving the selection of convenient (or good) pulse waves and then to apply it to a model programmed in said software to determine bgl.

The software may be configured to select an optimal sub-set of features resulting from the combination of said first and said second set of features through modelling as a sparse regularized optimization and applying greedy mathematical algorithms in order to characterize bgl.

According to another preferred embodiment, the set of pulse wave recordation may be collected during sleep of the subject. For example, a collection of pulse waves may be recorded at night when the subject is sleeping.

The pulse wave (PW) is a complex physiological phenomenon observed and detected in blood circulation. A variety of factors may influence the characteristics of the PW, including arterial blood pressure, the speed and intensity of cardiac contractions, and the elasticity, tone and size of the arteries. The circulation of blood through the vascular system is also influenced by respiration, the autonomic nervous system and by other factors, which are also manifested in changes in bgl. There are cardiovascular manifestations of bgl in healthy individuals as well those with a predisposition to diabetes.

Many of the features needed to analyze the PW for indications of levels of bgl can be taken by observing the contour of PWs over time. The typical PW shape is shown in FIG. 4. Generally, there are two main components of the PW in the time domain: the forward moving wave and a reflected wave. The forward wave is generated when the heart (ventricles) contracts during systole. The reflected wave usually returns in the diastolic phase, after the closure of the aorta valves. The returned wave helps in the perfusion of the heart through the coronary vessels as it pushes the blood through the coronaries.

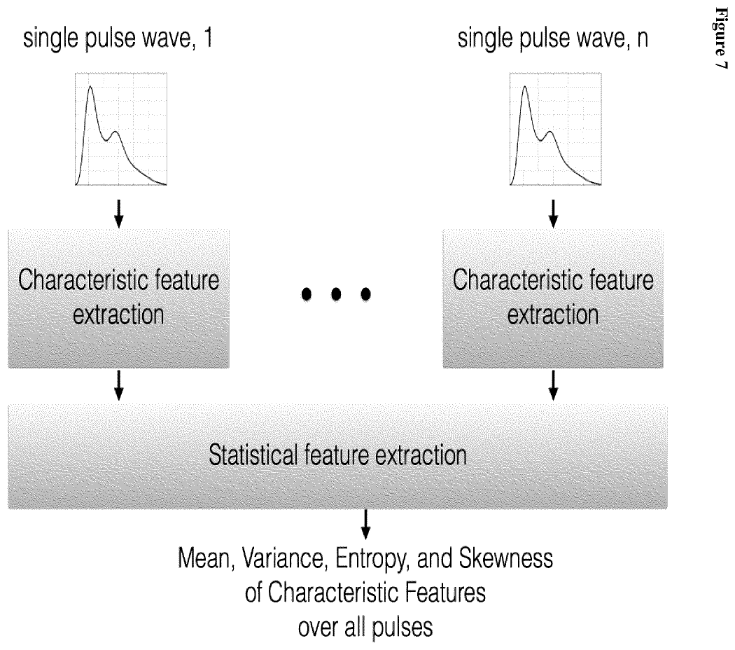

As noted in FIG. 6, 40 features can be identified and observed in this diagram. As a starting point, there is feature extraction taken directly from a point-based analysis of the PW timeline, which can provide seven PW points (that is, the five points specifically labeled in FIG. 4, as well as the start and end points). From these seven PW points, a group of features including amplitude, time, area and ratio may be derived. These may be referred to as the time and amplitude features where time denotes the distances between points on the PW and amplitude is the heights of the points calculated by measuring the distance between the lowest and highest points. There are also area-based features, where areas under various PW points are calculated and used to obtain additional PW features. Similarly, different areas under the same waveform can be compared in the form of ratios or other forms of statistical analysis. Ratios are also determined by dividing these features among themselves.

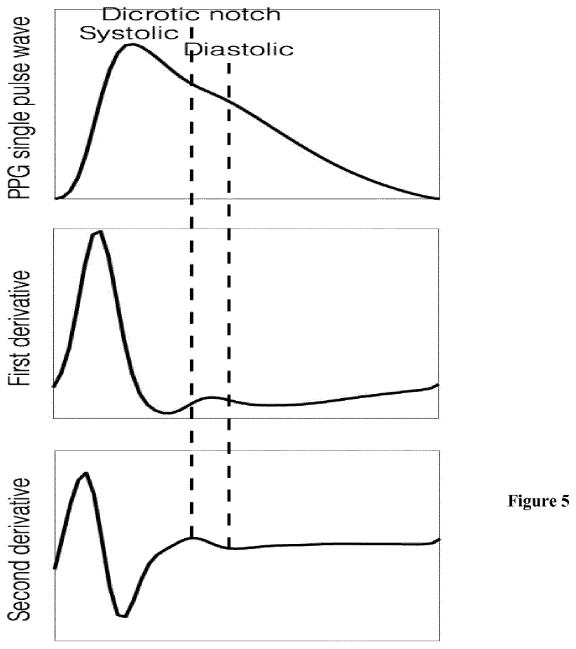

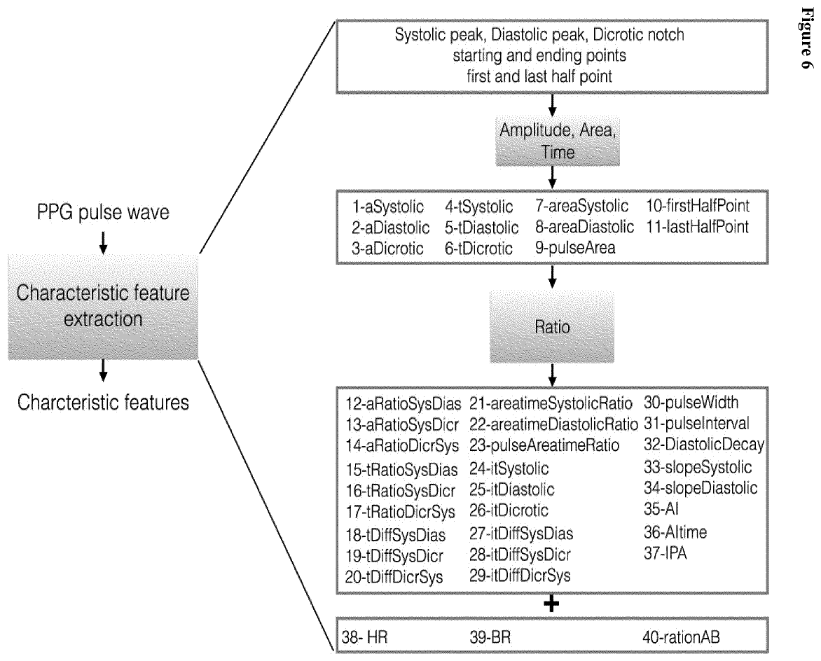

Besides the timeline basis of feature selection, the frequency domain is also a way of obtaining additional PW features. The Fourier transform among other methods transfers the signal from time domain to frequency domain, which shows how much of the signal lies within each given frequency band over a range of frequencies. By comparing the original waveform and the transform data, some special features can be detected in the frequency domain. The breathing rate, one of the features that is a part of the groups of selected features described previously, may be obtained from the frequency domain as the breathing rate is captured at a lower frequency than the PPG frequency. The heart rate can also be obtained by this methodology. For a further selection of features possibly useful for bgl, it is also helpful to evaluate the derivatives of waveforms. The first derivative of a PW leads to its local velocity (velocity pulse wave). To compute it, one can approximate it by a finite difference operator. This allows the precise analysis of sudden changes in the waveform and the identification of features, which may not appear on a timeline basis. The second-order derivative which is the derivative of the first derivative (acceleration pulse wave) is helpful in obtaining additional features for indications of bgl especially in cases where the timeline features are difficult to obtain as depicted in FIG. 5. All those collected features defined above are referred herein as the first set of features.

It is also helpful to use not only the features from the single PW (namely the first set of features) using these techniques but to also use the selected features in other statistical ways. For example, it may be of interest to see how the features change or evolve over time using, for example, additional parameters selected among mean, variation around mean and randomness and preferably selected among variability, variance, mean, standard deviations, entropy and skewness as noted in FIG. 7 as a third main step of obtaining additional parameters needed to estimate levels of bgl, referred herein as the second set of features.

It is therefore helpful to have collected at least two PWs and preferably several PWs (i.e. tens, hundreds or thousands thereof) over an extended duration to allow such comparisons. Through this statistical analysis, the behavior or patterns of change in features even ratios of changes not just absolute values or averages of specific features of the PW are analyzed: variances, which is a mathematical calculation of how spread out PWs points are from their mean; skewness is a way of quantifying the extend which a distribution of PW features differs from a normal distribution. An exemplary embodiment of the method may also include another statistical analytic method of obtaining PW features, which is entropy as an appropriate measure of randomness.

From these statistical analytic methods used on the PW features identified on the PW timeline, an exemplary embodiment of the method can extract and identify at least 160 features as noted in FIG. 7. This is done by using time, amplitude, area, and ratio to these PW features as identified in FIG. 6. Several additional features are identified including breathing rate and heart rate, which is the time between each pulse wave. Further, as illustrated in FIG. 7, all these features may then be used to statistically calculate their additional parameters selected among mean, variance, skewness and entropy to bring the total features used to 160 or more.

An exemplary embodiment of the method may also include a way of removing those features that have little or no correlation to changes in various bgl. The F-test or similar mathematical solutions using anova solutions are a means of narrowing down the number of features. An “F-test” is any statistical test in which the test statistic has an F-distribution under the null hypothesis. It is most often used when comparing statistical models that have been fitted to a data set, to identify the model that best fits the population from which the data were sampled. Through these statistical methods, the initial number of features can be reduced to around 70 features.

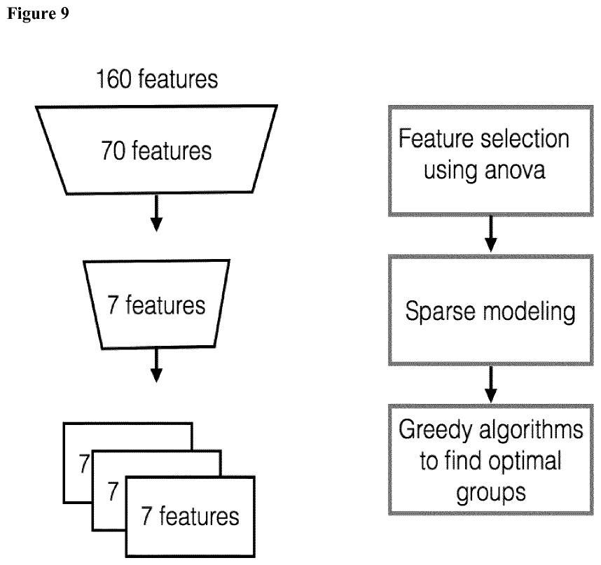

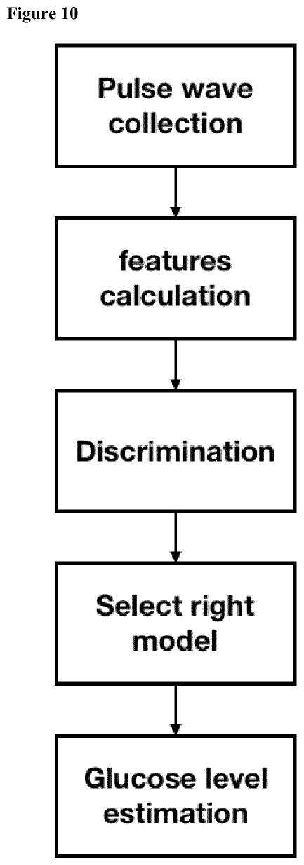

Since there may be some synergies between different PW features, an exemplary embodiment of a method may use a combination of features to identify correlations with bgl. Sparse mathematical methods are used to identify groups of features usually of no more than 7 features in each group. As illustrated in FIG. 9, the sparse technique or related technique is used to obtain around 20 groups of features. Through greedy or related mathematical techniques also illustrated in FIG. 9, each individual feature in each of these groups may be replaced one by one to identify the best or most indicative combination of features, which may be referred to herein as the optimal sub-set of features. These steps are repeated a few times until an optimal sub-set of features are identified. From this optimal sub-set or combination of features, algorithm(s) can be constructed either on a linear or nonlinear basis.

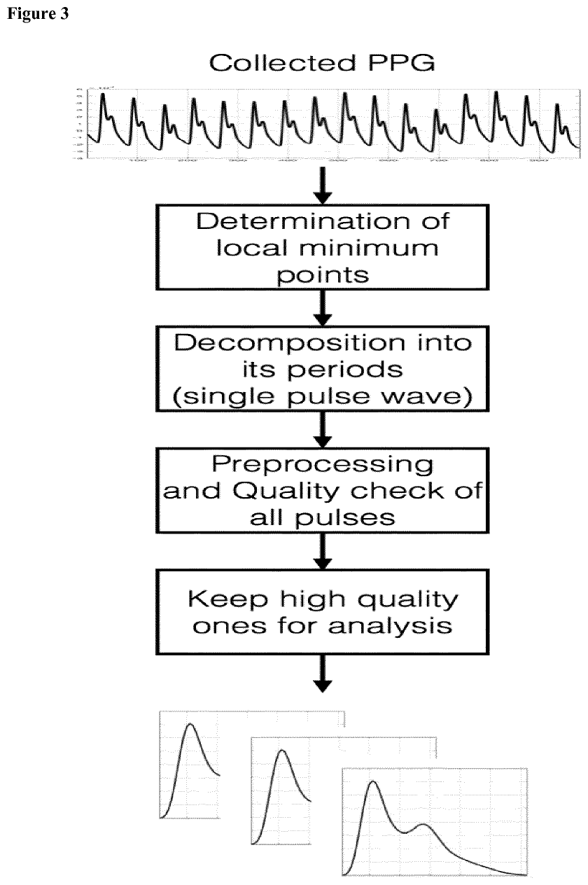

In an exemplary embodiment, pulse waves may be recorded beforehand. However, the pulse wave device according to an exemplary embodiment of a method can also collect blood pulse wave data for a period. Recordation of PWs can include several single pulse waves as shown in FIG. 3; according to the exemplary embodiment of FIG. 3, the raw data is sent to the processing module (software). The software first decomposes it into a set of single pulse waves by finding local minimum points of the main wave. After a quality check, good pulses or convenient pulses are selected. A “convenient or good pulse wave” is defined as the one that has a shape of a reasonable blood pulse and one can identify systolic and diastolic peaks plus the dicrotic notch point.

A single pulse wave may be denoted by p(t) where t presents the time coordinate. Then, the collected pulse is pₖ=(k Δt) where Δt denotes the sampling step with k=0, 1, 2, . . . , n. For example, assuming the subject heart rate is 60 beats/min, and the sampling rate of the device is 50 Hz then, in this example n=50, and Δt=20 ms. Note that first and second derivative of the pulse may be derived by using the finite difference method.

The pulse may be represented with a feature vector f=[f₁, f₂, . . . , f_{N}] where N is the number of features. To extract the characteristic feature vector for a single pulse, the following steps may be applied:
First, systolic, diastolic peak and dicrotic notch may be determined, plus the first and the last half points as shown in FIG. 4. The systolic peak is the first peak of the pulse (straightforward to find). The diastolic peak is the second one that can be more challenging to identify for some subjects (mostly for aged persons). If needed, in some exemplary embodiments, the first and the second derivative of the wave may be used to identify this point as illustrated in FIG. 5. The dicrotic notch may be the local minima point of the signal. This may also be identified using the first and the second derivatives of the signal as depicted in FIG. 5.
The time and amplitude values of the later discovered key points may be calculated. These may use the following notations: aSystolic, aDiastolic, aDicrotic, tSystolic, tDiastolic, tDicrotic (for amplitudes and times respectively).
The area under the curve is also computed by adding up a sampled points value multiplied by the sampling step. It is denoted by pulseArea.
The area under the curve is also divided into two areas, which may be distinguished by the dicrotic notch point. The first one is between the starting point and the dicrotic notch, which is called the systolic area under the curve, and the area under curve between the dicrotic notch and the ending point of the signal, which may be called the diastolic area under the curve. They are denoted by areaSystolic and areaDiastolic, respectively.
Normalizing the aforementioned area under the curves by the time period over which each one is calculated may yield the local cardiac output, which may in turn yield pulseAreatimeRatioSystolic, pulseAreatimeRatioDiastolic and pulseAreatime.
The time interval between the starting and the ending points may be called the pulse interval and denoted by pulseInterval.
The time interval between the first and the last half points may be called the pulse width and denoted by pulseWidth.
The time difference may be calculated between each two of the systolic peak, the diastolic peak and the dicrotic notch.
The time ratio may be calculated between each two of the systolic peak, the diastolic peak and the dicrotic notch.
The amplitude ratio may be calculated between each two of the systolic peak, the diastolic peak and the dicrotic notch.
The ratio of the areas may be calculated between the systolic area and the diastolic area.

In summary, first, the seven key points are identified (the systolic peak, the diastolic peak, the dicrotic notch, the first and last half and the ending and starting points). Then time, amplitude, and area linked to these points are computed. Then a generalized ratio may be defined, as shown in FIG. 7, which computes the ratio and the difference of two features and inverse of a given value. An example is shown in the ratio of the amplitude of the systolic and diastolic points, and of the time difference between systolic and diastolic points, as shown in FIG. 6.

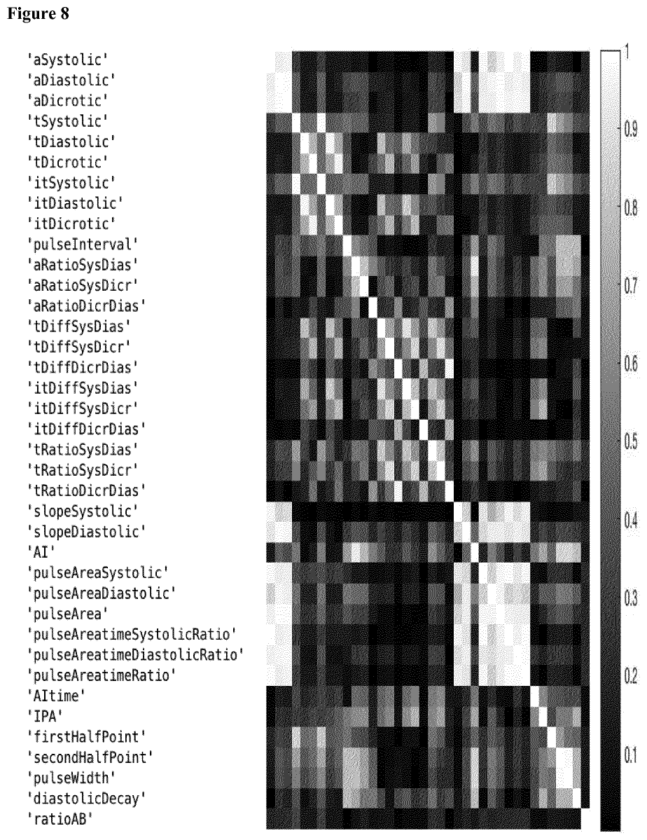

It is important to note that these characteristic features are complementary. To illustrate this, the correlation between each two features may be calculated by considering a data-set of blood pulse waves which includes 100,000 single pulses. FIG. 8 demonstrates the correlation image. The grayscale value is proportional to the correlation between the feature which corresponds to the row number and the feature which corresponds to the column number. In an exemplary embodiment, this may allow one to distinguish bgl by using only blood pulse waves.

After extracting the characteristic features also referred herein as the first set of features for each single pulse in a blood pulse wave, they may then be analyzed statistically by computing mean, variance, skewness and entropy for each feature over at least two ones as depicted in FIG. 7. These features are referred as statistical features. In some exemplary embodiments, characteristic and statistical features may be used and combined to distinguish bgl. Then, it is necessary to select an optimal combination of features referred herein as optimal sub-set of features and to determine an optimal model to compute bgl using the selected combination.

According to an exemplary embodiment, a model may be applied, [symbol] : X→ [symbol] , where x∈X is a pulse wave and/or a pulse wave feature vector and y∈ [symbol] is bgl. An optimal sub-set of features and an optimal model may be found by minimizing the loss function [symbol] ( [symbol] (x,a),y). The loss function measures how perfectly a model and a selected subset determine bgl. Upon the identification of this optimal model, the optimal model is then integrated into the software. After preprocessing the collected pulse waves and filtering out the good quality ones using the software, an important step in the software is to use the model to evaluate bgl. With this evaluation, the software can provide a form of visuals included in the software so that the users are able to observe in a user-friendly manner their respective bgl. Because of the computational aspects of the model, the software may be located on a larger computational device such as cell phones or mobile phones or computers or the clouds.

One can find the optimal sub-set of features and the optimal model using a brute force approach. This is a straightforward technique that goes through all possible sub-sets and finally selects the optimal one. As described, it requires high computation power. For example, in this case, it may be desired to find the optimal sub-set of features to quantify bgl. It is necessary to find what the number of features is, and which features they are. To use a brute force approach, it may be necessary to search through all different possible combinations of features. If there are more than one hundred features, then the solution space has more than 10¹⁴ elements. Therefore, it requires a significant amount of time to go through all sub-sets, find the model, and compute the loss function value for each one. Moreover, because of the complicated nature of the clinical study to collect data, typically there is not a large amount of data and as a result there is a high risk of overfitting.

To overcome these issues, the problem may instead be formulated as a regularized optimization one:

[symbol]

( [symbol] ,a)=argmin{ [symbol] ( [symbol] ,a,y)+ [symbol] (a)},

where [symbol] is the loss function that measures how well fit the model [symbol] to the measurement y, and the regularization term [symbol] includes the side information of the model for avoiding over-fitting. A first step of the framework has thus been determined. This may be demonstrated by a specific example:
The model [symbol] (in general, the model can be learned using machine learning techniques, can be linear or non-linear); it can be written in the form of:

[symbol]

(F)=Fa

Where a denotes a coefficient vector to describe the linear model [symbol] and each row of the matrix F is a pulse wave feature vector.
The loss function is a least square error, [symbol] (F,y)=∥Fa−y∥², where ∥⋅∥² is the [symbol] ₂-norm.
Sparsity regularization may be introduced by admitting [symbol] (a)=∥a∥₁ where ∥a∥₁=Σᵢ|aᵢ| with aᵢ is the i-th entry of the vector a.
A fast iterative shrinkage thresholding algorithm may be used to solve the later equation. The absolute value of the coefficient vector a may then be sorted. K features with the maximum absolute coefficient values may be selected. This step may be repeated for different regularization parameters, and the set which results in the least value of the least-square error ∥Fa−y∥² may be selected. After fitting the optimal linear model to the selected set of features, and after selecting an optimal combination of features from a sparsity point of view, greedy algorithms may be used in order to find an optimal solution, namely the optimal sub-set, but close to the sparse solution. Closeness from this point of view means to have the maximum intersection with the sparse solution.

A “greedy algorithm” is an algorithm paradigm to find the global optimum by finding a local one in each step. In the present example, a user may be looking for an optimal set of features with size seven to estimate or quantify bgl. They may start with an initial set which is the solution of the sparse representation. In each iteration, they may search for a group of local optimums such that new combinations differ with the last ones only in one feature (for example, 20 groups of feature combinations with seven features). This step may be continued up to the convergence criteria. Therefore, the advantage of a greedy algorithm is converging in a reasonable number of iterations prior to finding optimal groups; typically, finding the optimal solution requires many numbers of iterations using brute force techniques. But, it can converge to local optimums instead and the solution in this case may depend on the initialization. Initializing with the solution of the sparse representation leads to the optimal combinations and guarantees not facing over-fitting by choosing the minimum number of features.

In summary, the steps of selecting and making available to the users an optimum sub-set of features and optimal model for identifying bgl involve:
1. Using regularized optimization and sparse representation to select an optimal combination of features with the minimum size.
2. Using greedy algorithms initializing with the feature combination of the last step to select better combinations with the same number of features.
3. The selected subset of features combined with the optimal model is integrated into the software. After pulse wave collection, the software is then able to quantify the bgl using the optimal model and the optimal subset of features together with the pulse wave preprocessing step. The outcome of the software is then visualized in the form of a display or a set of numerical values.

One can improve the efficiency and the performance of the feature selection step by using F-test or anova to discard non-relevant features and then apply the aforementioned steps as shown in FIG. 9. After selecting the optimal features, one can use a different learning approach for the final decision steps (finding the model). One simple model, which can be used in one exemplary embodiment, can be a linear model. Other examples, which may be used in other exemplary embodiments, include an artificial neural network, support vector machine, non-linear and polynomial models.

With the optimal group of features identified and an algorithm(s) designed to best use this group of features, the mathematical model can be built into the software or app used to identify and quantify the bgl. In some exemplary embodiments, these calculations can be contained in the software located on a device such as a mobile phone or computer, or can be in a cloud form, which, in turn, may be available to the user for example on the user's pulse wave device.

It is necessary to identify pulse wave features or groups of pulse wave features that are the most informative to changes in bgl. This process involves eliminating those features or groups of features that correlate closely to both bgl changes and to other phenomena that are related to bgl changes. For instance, changes in bgl are related to food intake and different types and quantities of food consumption. The digestive process involves muscle contractions and other bodily functions that affect blood flow. In order to identify pulse wave features directly related to bgl changes, it is necessary to not include those pulse wave features that correlate to metabolism and other factors related to eating. These pulse wave features should not be included as they correlate to bgl changes regardless of effects of changes in sugar levels in the blood. This is done by empirically examining and identifying pulse wave features that change with different quantities and types of foods consumed. These features should generally not be included in the selected groups of pulse wave features used to determine bgl.

In another exemplary embodiment, the invention provides a pulse wave device for quantifying the blood glucose level in a subject and/or for discriminating between different sources of blood glucose level changes, wherein blood glucose level changes are selected among the type of nutrients, type of sport activities, type of stresses and/or fatigue or a combination thereof, said pulse wave device being applied on a pulse-taking location on the body of said subject; said pulse wave device comprising:
a sensor module (1) for collecting information data from the pulse wave, a memory module (4) for storing the pulse wave information data on the pulse wave device, a display module (3) for displaying the results of the blood glucose level and/or the discrimination between said different sources of blood glucose level changes and a processor module (2) comprising:
means of extracting and selecting from each single pulse wave and from its first and second derivation a first set of features providing information data consisting in the time, amplitude, area, ratios, heart rate and breathing rate;
characterized in that, said processor module (2) is configured to perform a statistical analysis on the collected information data from the pulse wave and/or on said first set of features obtained from at least two single pulse waves to arrive at a second set of features providing additional information data consisting in the mean, variation around the mean, randomness and/or time series analysis between said first set of features of the at least two single pulse waves; and wherein, said processor module (2) further comprises means for combining said first and second set of features and means to analyze and display the results of the blood glucose level and/or the discrimination between said different sources of blood glucose level changes of said subject.

According to an embodiment of the invention, the pulse wave device is further adapted to identify diabetic or pre-diabetic subjects from healthy subjects and wherein diabetes or pre-diabetes comprises Type I diabetes, Type II diabetes, hyperglycemia impaired fasting glucose and impaired glucose tolerance.

Preferably, time series analysis are performed by ANN, RNN, DL or CNN techniques.

The processor module (2) comprises a software adapted or configured to calculate the pre-selected combination of features after the preprocessing step involving the selection of convenient or good pulse waves and then applies it to the model programmed in the software to determine or quantify the bgl. According to an exemplary embodiment, the software is configured to calculate a pre-selected combination of said first and second set of features after a preprocessing step involving the selection of convenient (or good or clear or suitable) pulse waves and then to apply it to a model programmed in said software to quantify the bgl.

The software or app may be configured to select an optimal sub-set of features resulting from the combination of said first and said second set of features through modelling as a sparse regularized optimization and applying greedy mathematical algorithms to measure bgl.

In an exemplary embodiment, the pulse wave device may be adapted for personal health care diagnosis. This invention is to include the providing of additional information that can help the user better manage bgl. Some of this data may be collected digitally from other sources and be transmitted into the device or app to help this monitoring process. Other data may be added manually with fields in the app available for manual input of data or comments. This may include data related to sleep, stress, and physical activity. The app or device or software will also include the ability to log in manually other related data that may be helpful in improving patient outcome as it related to controlling bgl. Regular comments on diet, calorie intake, types of foods eaten can be included here. This is a way to gather information in one place related to bgl control and can also serve as a means of encouragement in applying life style choices to better bgl control. Regular user input with regular feedback is known to help with compliance and with improved patient outcomes. In addition, the app may include the physiological characteristics of the user such as age, weight, body mass index, and other factors, which may help improve the measurements and understanding of the bgl.

According to an exemplary embodiment, the means of extracting pulse wave signal namely the sensor module (1) for collecting information data from the single pulse may be selected among pulse-taking sensors, photo or video imaging, optical emitters based on LEDS or a combination thereof.

In an exemplary embodiment, the pulse wave device may be deprived of a filter that distorts the pulse wave shape.

Heart-generated pulse waves propagate along the skin arteries, locally increasing and decreasing in blood volume with each heartbeat. The dynamic blood volume changes in relation to the heart function, size and elasticity of blood vessels and various neural processes. Blood absorbs lighter than the surrounding tissue. Therefore, a reduction in the amount of blood is detected as an increase in the intensity of the detected light and vice versa. Photoelectric Plethysmography (PPG), which measures the degree of light absorption in a tissue based on the change in this peripheral blood flow rate, is an optical method of measuring pulse waves. Currently, this is the most popular means of acquiring pulse wave data. Other means are also available and may increase in popularity in the future.

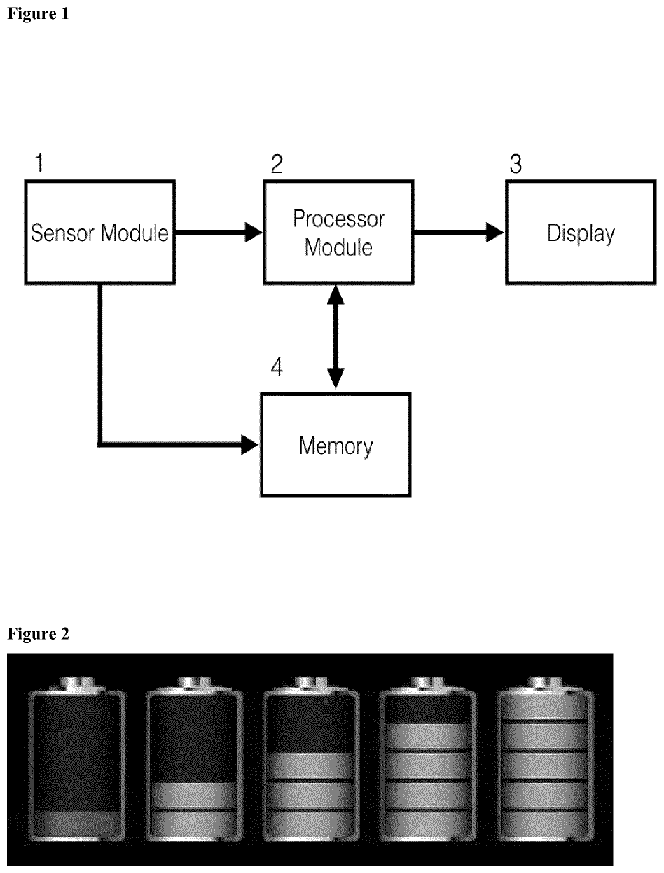

Also referred to as pulse oximeters, the PPG hardware consists primarily of the following main components as shown in FIG. 1. A sensor module (1) for collecting information data from the pulse wave, a memory module (4) for storing the pulse wave information data on the pulse wave device, a display module (3) for displaying the results of the bgl and a processor module (2) comprising a software.

Processor module (2) may take a variety of forms, such as a desktop or laptop computer, a smartphone, a tablet, a processor, a module, or the like. Processor module (2) may represent, for example, computing or processing capabilities found within desktop, laptop, notebook, and tablet computers; hand-held computing devices (tablets, PDA's, smart phones, cell phones, palmtops, smart-watches, smart-glasses etc.); mainframes, supercomputers, workstations or servers; or any other type of special-purpose or general-purpose computing devices as may be desirable or appropriate for a given application or environment. Processor module (2) might also represent computing capabilities embedded within or otherwise available to a given device. For example, a Processor module (2) might be found in other electronic devices such as, for example, digital cameras, navigation systems, cellular telephones, portable computing devices, modems, routers, WAPs, terminals and other electronic devices that might include some form of processing capability. Processor module (2) might include, for example, one or more processors, controllers, control modules, or other processing devices, such as a processor. Processor module (2) might be implemented using a general-purpose or special-purpose processing engine such as, for example, a microprocessor, controller, or other control logic.

Processor module (2) might also include one or more memory modules (4), simply referred to herein as memory module (4). For example, preferably random access memory (RAM) or other dynamic memory, might be used for storing information and instructions to be executed by processor module (2). Memory module (4) might also be used for storing temporary variables or other intermediate information during execution of instructions to be executed by processor module (4).

As used herein, the term “module” might describe a given unit of functionality that can be performed in accordance with one or more embodiments of the present application. As used herein, a module might be implemented utilizing any form of hardware, software, or a combination thereof. For example, one or more processors, controllers, ASICs, PLAs, PALs, CPLDs, FPGAs, logical components, software routines or other mechanisms might be implemented to make up a module. In implementation, the various modules described herein might be implemented as discrete modules or the functions and features described can be shared in part or in total among one or more modules. In other words, as would be apparent to one of ordinary skill in the art after reading this description, the various features and functionality described herein may be implemented in any given application and can be implemented in one or more separate or shared modules in various combinations and permutations. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

In this document, the terms “computer program” and “software” and “app” are used to generally refer to transitory or non-transitory media such as, for example, memory module (4), storage unit, media, and channel. These and other various forms of computer program may be involved in carrying one or more sequences of one or more instructions to a processing device for execution.

FIG. 1 is a representation of an exemplary embodiment of a pulse wave device. This PPG probe includes one or several infrared light-emitting diodes (LEDs) and/or green or other color LEDs and one or several photodetectors.

Many combinations of these two main components are possible to try to best obtain pulse wave signals for as many different human physiological factors as possible such as pigmentation in tissue, venous configuration, bone and other features than can vary from person to person and body location (wrist, finger, ear, arm, etc.). The light sources from the optical emitters are LEDS which illuminate the tissue and the photodiodes which are photodetectors used to measure the variations in light intensity associated with the changes in blood vessel blood volumes. The array of sensors is designed to allow multiple colors, wavelengths, light angles, and distances between sensors to best characterize and acquire the pulse waves. This array of sensors is connected through an electronic circuit board to the memory unit and battery. In this system, according to some exemplary embodiments, operational amplifiers may be used to amplify the signals, and high-resolution analogue-to-digital converters may also be used. Bluetooth is used to send the data to a larger computing device such as a mobile phone. The device also includes a mini USB to permit manual transmissions of data. In the case of an app, Bluetooth, mini-USB can allow the transmission of data into the app from other data sources outside the smart phone.

A variation on this optical sensor pulse wave acquisition is using photo or video imaging, also referred to as video plethysmography. It is also possible to capture pulse waves by taking either photos or a series of photos, which may be of a contact type for short-distance measurements (for example, this may require a user to place their finger on a mobile phone camera to use the phone camera LED light) or may be of a non-contact type for longer-distance measurements, or require the camera to be aimed at the face or other parts of the body where it is also possible to capture pulse waves. One embodiment of the invention is to develop an app downloadable to a smart phone. The app would have direct access to data generated from a smart phone camera and use the processing power of the smart phone to extract, process, calculate and visualize bgl directly on the smart phone, thereby avoiding the need for additional hardware.

According to an exemplary embodiment, some form of hardware may be used to capture quality pulse wave signals regardless of whether they are obtained from optical sensor technologies as described or from photo or visual imagery and/or any other means of obtaining a clear pulse wave, preferably the raw signal, which may allow the different pulse wave features to be distinguished. Accurate and reliable presentation of the pulse waveform is of importance. Other methods of acquiring the pulse wave may be contemplated in other exemplary embodiments.

In some exemplary embodiments, software may allow for acquiring, collecting, analyzing and displaying the analysis and interpretation of the pulse wave data in a user-friendly manner. The pulse wave device may have inbuilt firmware to ensure the smooth running of the components including the operation of the sensors and the handling and storage of acquired pulse wave data. The pulse wave device may also permit the transfer of the acquitted data to a larger computer processing device such as a mobile phone or computer.

Once the data is correctly transferred to a desired computer platform such as a mobile phone, apps such as mobile apps allow for further computation and provide the user with a good user experience. This includes good visuals so the user can quickly understand their bgl without being experts in the field. In an exemplary embodiment, the data may be security protected to ensure privacy.

According to an exemplary embodiment, the processor module (2) (comprising a software) related to envisioned device is configured to calculate a pre-selected combination of said first and second set of features after a pre-processing step involving the selection of convenient (or good) pulse waves and then apply it to a model programmed in said software to determine bgl.

In a number of instances where this model determines that the subject falls outside the predetermined desired bgl, the processor module (2) will alert the subject from the device linked to a mobile phone or other device with a display of this occurrence. This warning or alert can take the form of an alarm noise or as text or symbol display on a screen.

Preferably the warning unit can alert the subject when a certain level of bgl falls outside the levels desired.

A necessary step in the pulse wave device is to collect the pulse wave data using the described or similar biosensor device or any type of device that can collect and register pulse waves.

Generally, pulse waves can be obtained from many parts or pulse-taking location of the body where there is access to pulses (wrist, finger, arm, ear, head, etc.). In an exemplary embodiment, the sensor may be configured to fit snugly against the chosen part of the body to avoid gaps between the sensor and the tissue. Biosensors in ear buds have, for example, a considerably different shape from a wrist-based location, which is more of a 2-dimensional surface. If light gets in between the sensor(s) and the skin this will distort the pulse wave signals from ambient light, ranging from direct sunlight to flickering room light. A finger tip pressed against a smart phone camera lens can also serve this picture as well as a camera aimed at various parts of the body including the face to get pulse wave signals.

Pulse taking locations vary in vascular structure, which affect rates of blood perfusion as lower perfusion correlates with lower blood flow signals. The pulse wave shapes need to be considered since they can be different depending on the location of data collection. The pulse should also be taken, for example in an area where the artery is less likely to move as well as in an area where other movements such as muscle, tendon and bone can, if possible, be minimalized to avoid unnecessary noise artefacts.

It may further be noted that data collection may be better when taken lying down or sitting to avoid abrupt body movement; however, it may also be noted that this is not required. Movements will cause motion artefacts, which can distort the signal quality. The fewer the number of artefacts, the less that needs to be done to filter out the noisy elements in the signal. For example, according to an exemplary embodiment, pulse waves may be measured during the night when the subject is asleep. This limits light and motion artefacts and permits a long period of data acquisition without requiring behavioral changes on the part of the subject. Overnight data collection is also valuable in that the data captured reflects the physiological changes due to the day's activities. A longer sample period also permits more accurate data analysis since erroneous data can be discarded as there are plenty of other pulse wave samples to choose from. It is therefore helpful to have collected at least two PWs and preferably several PWs (i.e. tens, hundreds or thousands thereof) over an extended duration to allow good comparisons.

A longer data collection period also allows for pulse wave features to be analyzed in terms of variance and variability. Often pulse wave analysis relies on absolute pulse wave features based on averages and means or even through the comparisons of single pulse waves. Having a larger data base of pulse waves over an extended period allows the analysis of the changes in pulse wave features through such additional variables as variance, variability and skewness. This is also helpful when machine learning and other mathematical techniques are applied where generally larger databases are needed.

To derive indications of bgl, according to an exemplary embodiment, a pulse wave analysis may be performed using the full contours and features present in in a pulse wave, preferably an unfiltered pulse wave. Many pulse wave acquiring devices as described above use filters that distort the pulse wave shape to highlight the heart rate peaks. This is because the main objective of the device is to measure heart rates and the derived HRV. Filters are also used to remove environmental effects and other disturbances, which can change the morphology of the pulse wave. It may instead be desired to use raw pulse wave data; this data can be acquired either directly without signal manipulation or by removing the filters from the acquired filtered PPG signals. Reverse filters can also be applied. The acquired signals need to be examined to ensure clear pulse wave contours are obtained (herein defined as convenient pulse waves). Bad or distorted PPG signals need to be either corrected or discarded. Since there are lots of pulse waves in a sample, according to an exemplary embodiment, this may be accomplished through a program that “de-bugs” the signals by taking the bad signals out from the good ones. This part of the sensor system includes “signal quality flags”, generated via signal processing, to indicate the quality of the biometric data and to inform the program to exclude low quality and erroneous data.

With the optimal sub-set or group of features identified and an algorithm(s) designed to best use this group of features, a bgl may be identified (including machine learning). In some exemplary embodiments, a mathematical model can be built into the software or app used to determine bgl. These calculations can be contained in the software or app located on a device such as a mobile phone or computer or it can be in a cloud form, which, in turn, is available to the user on the user's pulse wave device.

In an exemplary embodiment, it may be desired to display a clear visual in the form of, for example, a gauge or graph depicting the level of bgl (see FIG. 2). A variation on this visual is to indicate a numerical value in a range of, say, 1 to 10. For users of the pulse wave device that seek more detailed information on their pulse, the device may include the ability to obtain data of considerably more detail such as more specific aspects of bgl including such related data as sleep data, physical activity tracking/data and logs of daily comments such as food consumption. The values of specific features or combination of features may also be indicated. The device is designed to also provide data on how the calculations are derived as well as provide bgl related indications for other health related web sites.

In an embodiment of the invention, the first set of features is determined by measuring the entire pulse wave timeline, or by identifying a set of pulse wave points selected among the systolic, diastolic, dicrotic notch, the first and last points corresponding to the half-height of the systolic peak and the starting and ending points of said single pulse wave.

In accordance with the invention, the ratios in said first set of features comprise:
A ratio of an amplitude of a systolic peak and an amplitude of a diastolic peak;
A ratio of the amplitude of the systolic peak and an amplitude of a dicrotic notch;
A ratio of the amplitude of the dicrotic notch and the amplitude of the diastolic peak;
A ratio of a time value of the systolic peak and a time value of the diastolic peak;
A ratio of the time value of the systolic peak and a time value of the dicrotic notch;
A ratio of the time value of the dicrotic notch and the time value of the diastolic peak;
A time difference between the time value of the systolic peak and the time value of the diastolic peak;
A time difference between the time value of the systolic peak and the time value of the dicrotic notch;
A time difference between the time value of the dicrotic notch and the time value of the diastolic peak;
A local cardiac output corresponding to a ratio of an area under the curve to a time difference between a starting time and an ending time;
A ratio of the area under the curve between the starting point and the systolic peak to the amplitude of the systolic peak;
A local systolic cardiac output corresponding to a ratio of an area under the curve between the starting point and the dicrotic notch to the time value of the dicrotic notch;
A ratio of an area under the curve between the starting point and the dicrotic notch to the amplitude of the systolic peak;
A local diastolic cardiac output corresponding to a ratio of an area under the curve between the dicrotic notch and the ending point to the time difference between the time value of the dicrotic notch and the time value of the ending point;
A ratio of an area under the curve between the dicrotic notch and the ending point to the amplitude of the diastolic peak;
A pulse width at ten, thirty, fifty, seventy, or ninety percent corresponding to a time difference between the first and the last points corresponding ten, thirty, fifty, seventy, or ninety percent of the systolic peak, respectively;
A time difference between the first point corresponding to ten, thirty, fifty, seventy, or ninety percent of the systolic peak and the systolic time;
A time difference between the systolic peak and the last point corresponding to ten, thirty, fifty, seventy, or ninety percent of the systolic peak;
A pulse interval corresponding to the time difference between the ending and starting time;
A slope of the systolic peak corresponding to the ratio of the amplitude of the systolic peak by the time value of the systolic peak;
A slope of the diastolic peak corresponding to the ratio of the amplitude of the diastolic peak by the time difference between the ending point and the diastolic peak;
A diastolic decay corresponding to a logarithm of the slope of the diastolic peak;
An inflection point area ratio corresponding to the ratio of the area under the curve between the dicrotic notch and the ending point divided by the area under the curve between the starting point and the dicrotic notch;
An augmentation index, corresponding to the ratio of the amplitude of the systolic peak divided by the amplitude of the diastolic peak;
the ratio of the local diastolic cardiac output by the local systolic cardiac output, or the inverses thereof;
A pulse mean corresponding to the mean of the pulse curve;
A pulse standard deviation corresponding to the standard deviation of the pulse curve;
A pulse median corresponding to the median of the pulse curve;
A ratio of the local systolic cardiac output and the local diastolic cardiac output;
A ratio of the amplitude of the systolic peak minus the amplitude of the dicrotic notch divided by the amplitude of the diastolic peak minus the amplitude of the dicrotic notch;
A ratio of the area under the curve between the systolic peak and the dicrotic notch to the time difference between the time of the systolic peak and the time of the dicrotic notch;
A ratio of the area under the curve between the systolic peak and the dicrotic notch to the amplitude of the systolic peak.

In accordance with the invention, the variation around the mean in said second set of features consists of skewness, variance, standard deviation and power spectrum.

In accordance with the invention, the randomness in said second set of features consists of entropy.

According to an embodiment of the invention, the processor module (2) is configured to calculate a pre-selected combination of said first and second set of features after a preprocessing step involving the selection of convenient pulse waves and then to apply it to a model programmed in said processor module (2) to determine bgl.

Preferably, the processor module (2) is configured to select an optimal sub-set of features resulting from the combination of said first and said second set of features through modelling as a sparse regularized optimization and applying greedy mathematical algorithms in order to obtain bgl.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein. Various references are cited throughout this specification, each of which is incorporated herein by reference in its entirety.

The foregoing description will be more fully understood with reference to the following Examples. Such Examples, are, however, exemplary of methods of practicing the present invention and are not intended to limit the scope of the invention.

### EXAMPLES

### Example 1 (AI and Glucose Level)

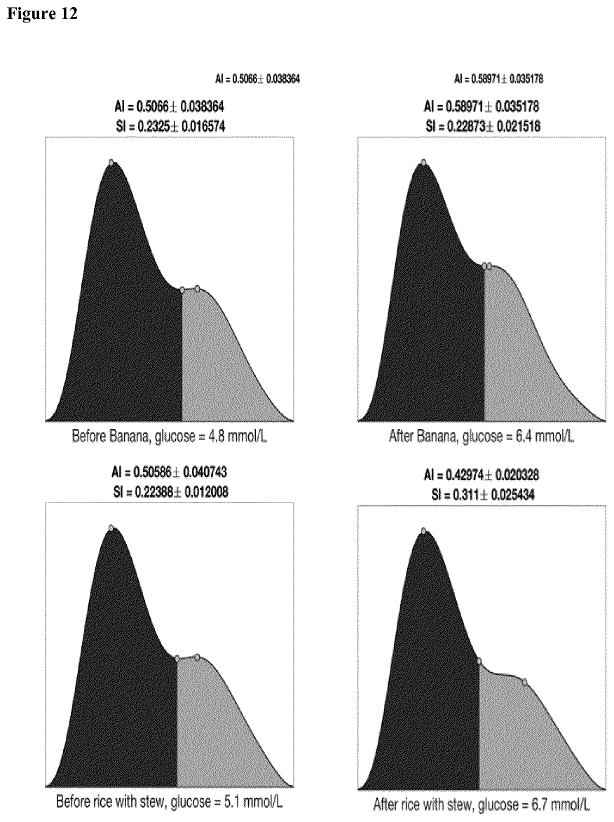

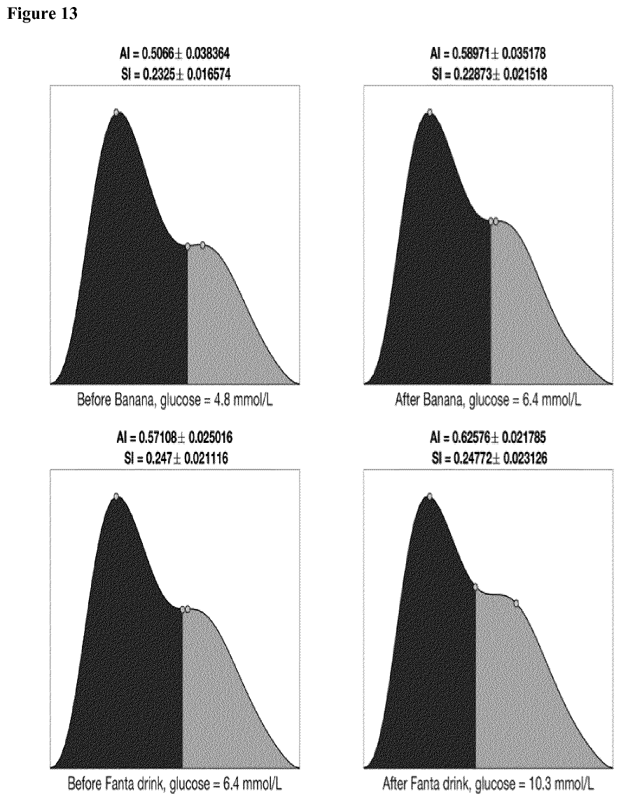

A study was performed on seven subjects for four days where the subjects ate bananas and rice and stew (including red meat and vegetables) to investigate the relationship between glucose level and (AI/SI). SI's and AI's are measured 5 min after intake as are the glucose levels using a glucose measuring device where a blood sample is taken with each test. The same process is done for another four days, while the subjects eat 400 gr banana. In both cases glucose levels increase, while AI decrease after eating rice and stew and AI increases after eating banana. This is also shown before and after eating rice with stew. There were no significant changes in SI values. As shown in FIG. 12 and FIG. 13, AI increases after eating banana and AI decreases after eating carbohydrates and fats while in the both cases glucose levels increase.

### Example 2 (AI and SI are not Suitable for Glucose Level Estimation)

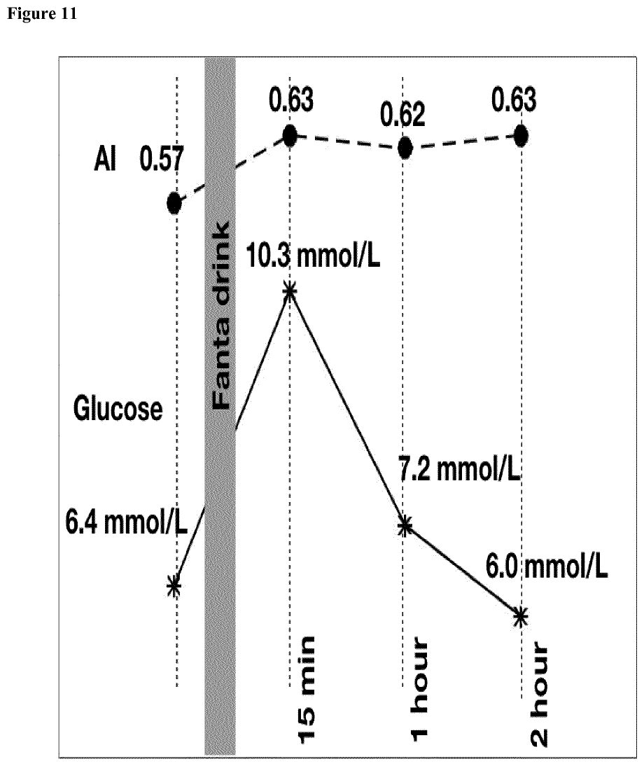

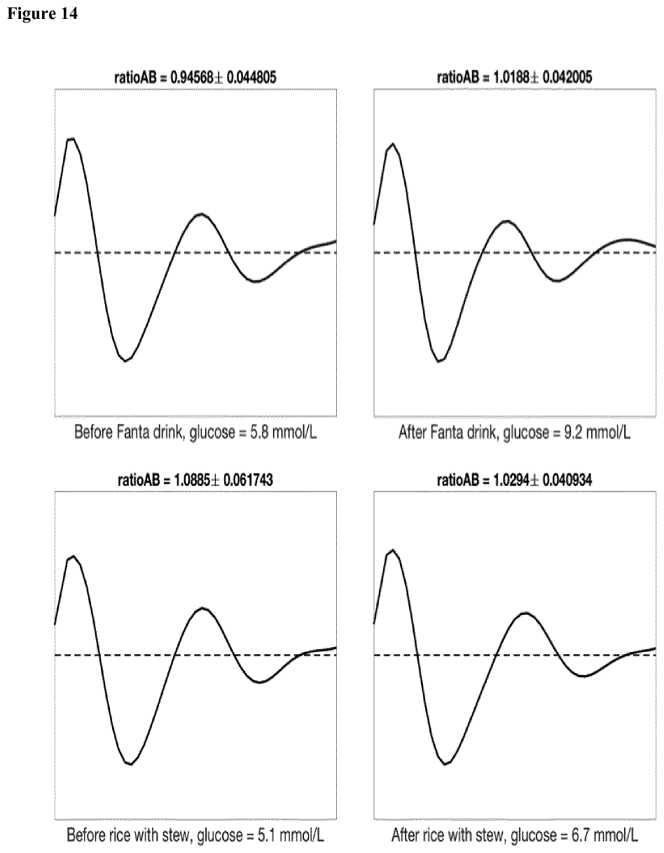

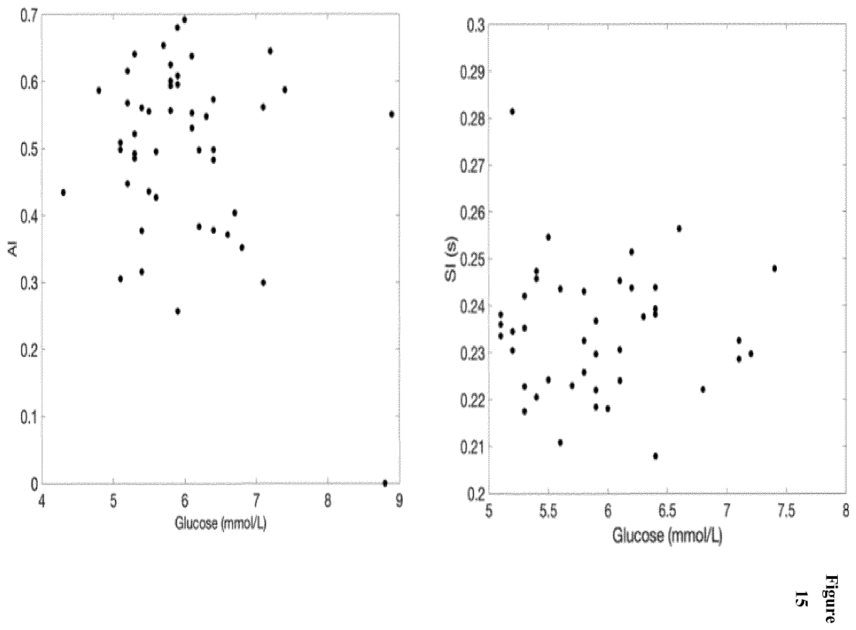

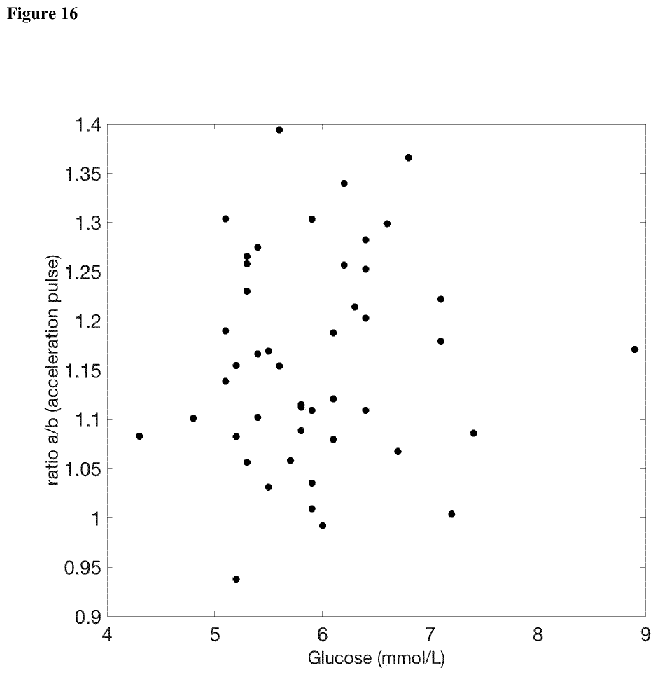

A similar study (as illustrated in FIG. 11) was done with the subjects drinking Fanta. While glucose levels spike up, the AI levels rise only moderately and plateau after two hours even through bgls go back to roughly the same levels prior to the drink. The scatter plots in FIG. 15 illustrate the same lack of pattern between AI and SI and bgl as does FIG. 14 using the accelerated pulse wave. The subjects were asked to drink one bottle of 500 ml Fanta and take the glucose blood test as well as monitor the subject's pulse waves similar to the prior described study also for four days. As shown in FIG. 14, after the glucose drink, blood glucose level and AI both increase. After two hours, even though the bgl return back to the same or similar value. The AI and SI remain high. However, the AI levels increased at significantly lower rates. SI remained relatively stable.

In both these examples, after the Fanta drink and after rice and stew, the ratio of the first and the second peak of acceleration pulse wave increase has totally opposite behavior as shown in FIG. 14.

In EP 3 289 968 A1, the inventors claimed that when glucose level increases, AI decreases. This study shows that it is not the case after drinking a soda drink.

### Example 3 (There cannot be a Linear Relationship Between Glucose Level and AI/SI)

In a study performed at the Cantonal Hospital of Vaud (CHUV), Applicants did a study of 8 students during exercise and rest over a 4-hour period. Independent of food, the typical bgl varied by over 20%. This is because the body uses glycogen for energy. With heightened exercise, insulin concentrations tend to decline, and plasma glucagon shows a gradual increase. This increase glucose levels availability to the cells, maintaining adequate glucose concentrations to meet increased metabolic demands. These changes are also affected by intensities of exercise.

In EP3269305 A1, the invention discusses the use of an “accelerated pulse wave” or commonly referred to as the second derivative. Changes in the inflection points of the pulse wave are better visualized using the second derivative allowing a more accurate calculation of the peaks and notches as per changes from the baseline. The AI and SI are often calculated from the acceleration pulse wave. The heights of these main inflection points are used for analysis.

The inventors in this patent application claim that acceleration pulse wave is correlated with the glucose level, which is not the case in general for example after drinking a gluco drink.

Accurate correlations from these identified pulse wave features provide limited results as they do not consider other physiological changes that occur in the body other than changes in blood sugar level other than food intake. Effectiveness of measuring bgl will vary based on circumstances. Exercise has an influence on bgl.

Many studies show that people usually report a higher heart rate after eating particularly carb heavy meals. Following the consumption of food, the body directs blood flow to the digestive tract to assist with digestion. This can lead to a faster heart rate especially as the workload picks up from the chemical processes and other metabolism mechanisms. Accordingly, pulse wave features related and including the heart rate such as the augmentation index are correlating after meal effects due to metabolism as much as they are tracking bgl.

Sleep and stress have also an impact on bgl in a disproportional amount to food influences. Studies repeatedly show that too little sleep is associated with higher bgl and greater insulin resistance. These three additional factors: exercise, sleep and stress will increase or decrease in disproportional rates to such pulse wave measurements as AI, SI in heart rates and heart rate variability.

For these reasons, these prior art documents are not able to correlate pulse rates or pulse waves accurately enough to measure bgl. The correlations exist but they are not accurate enough as described. Other pulse wave features other than those few cited in this prior art is needed.

Accuracy is especially important to avoid health problems related to abnormal bgl and to help improve patient outcomes for those undergoing therapy or trying to better adapt life style choices to improve bgl control.

### Example 4 (Metabolism Versus Glucose Level Variation)

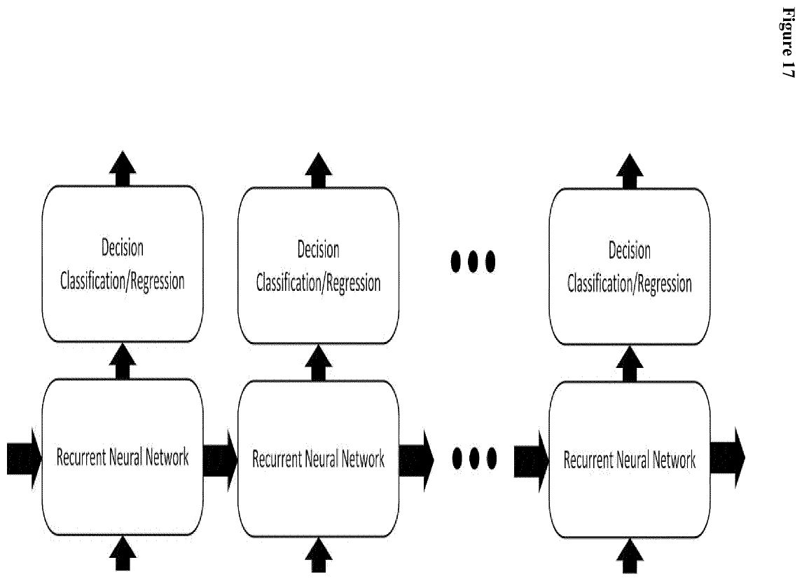

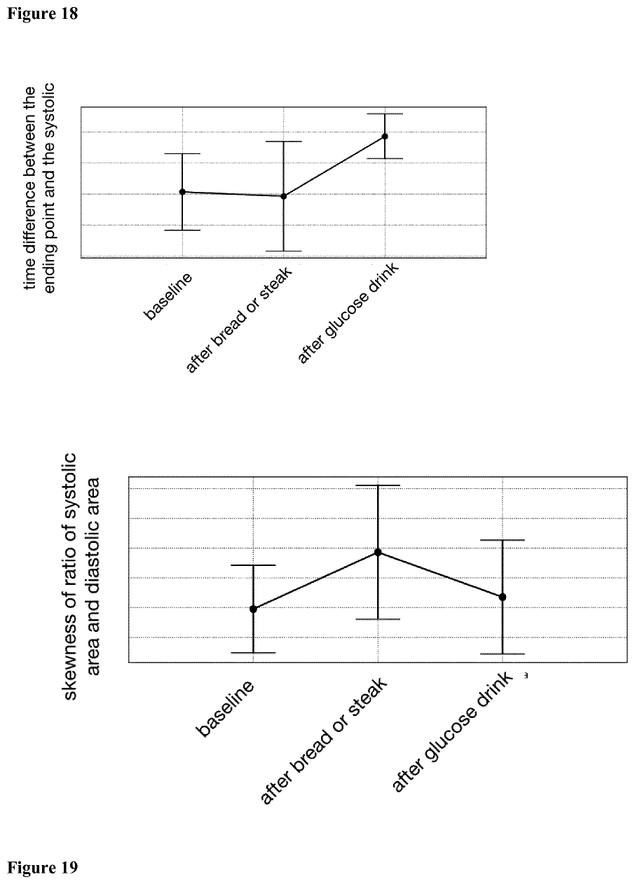

A challenge in estimating blood glucose levels after eating is the impact of the digestive process and the metabolism on the collected pulse wave. To address this, Applicants compared the effect of a glucose drink that is high in glucose and usually requires a lower metabolism in contrast to the effect of steaks and bread that require higher metabolism and lower sugar level on seven healthy individuals. Each person performed the test three times for each of glucose drink, steak, and bread. The protocol test was that in the morning, before eating anything, their blood glucose level was measured, and also, their pulse waves were collected for two minutes. Then depending on the protocol, the tested subjects drunk 500 ml glucose drink, or they ate 400 gr bread or 300 gr steak. After doing this step, their glucose level were measured again along with the collection of their pulse waves for two minutes. After analyzing the pulse, Applicants found a model based on different group of features that could separate the two processes. For example, the skewness of the ratio of systolic area by diastolic area showed a major change after bread/steak (high metabolism, low sugar level), but almost no noticeable change after glucose drink (low metabolism, high sugar level) as shown in FIG. 18. On the other, the time difference between the ending point and the systolic time behaved quite differently as depicted in FIG. 19. It is worth mentioning that the final model combines a group of features to improve the accuracy of discriminating between the effect of metabolism process on the collected pulse wave and the impact of the blood glucose level variations.

### Example 5 (Non-Invasive Glucose Monitoring in Diabetic Patients)

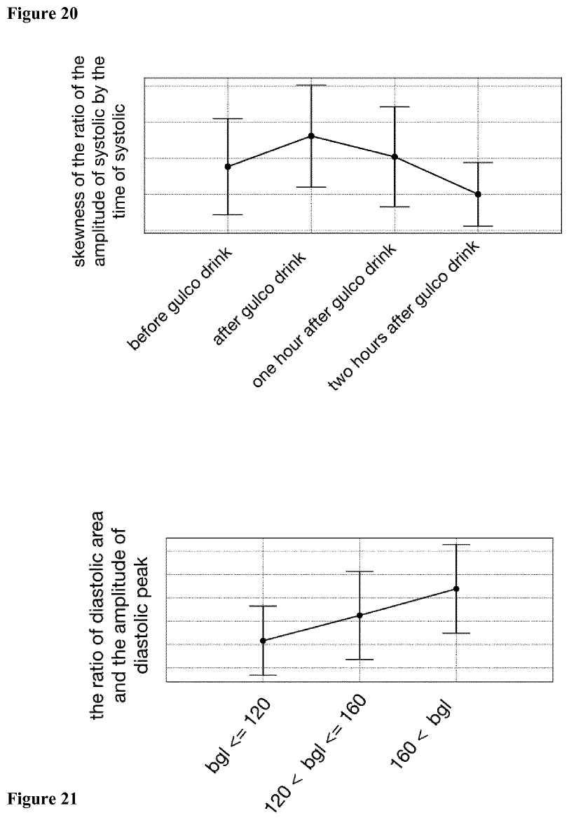

One special point of importance of the invention (non-invasive blood glucose monitoring) is for diabetic patients who need to monitor their blood sugar regularly. For this reason, Applicants conducted a study on seven diabetic patients in the age group between 60 to 70 years old. Applicants monitored them for fifteen days. Applicants didn't interfere with the subject's daily schedule. Each subject measured their glucose level before and after breakfast, lunch and dinner using a medical invasive device. In addition, their pulse waves were collected for two minutes at the same time of glucose monitoring. Applicants analyzed the data to find a model to estimate the glucose level based on a group of features derived from the collected pulse waves. One of the features as depicted in FIG. 20 was the ratio of diastolic area and the amplitude of the diastolic peak. It is worth mentioning that the final model combines a group of features to improve the accuracy of non-invasive monitoring on the blood glucose level.

### Example 6 (Recovery Pattern of Blood Glucose Level after a Glucose Drink)

As explained in the present invention, one of the challenges of finding a model for estimating blood glucose levels is that different factors can affect the glucose concentration in the blood. In this study, Applicants wanted to investigate the effect of glucose drink and monitor its recovery pattern. Research has shown that it usually increases sharply after glucose drink, but can drop significantly after two hours. To address this issue, Applicants conducted a study on five healthy subjects. Each one tried a bottle of 500 ml glucose drink for five different days. In each day, their glucose level was measured and also, their pulse wave were collected in the morning before eating or drinking anything. Then, the teste subjects drunk a bottle of 500 ml glucose drink, and the same measurements were made after the drink, one hour and two hours later. Between measurements, they were not allowed to eat or drink. As depicted in FIG. 21, the skewness of the ratio of the amplitude of systolic by the time of systolic was highly correlated to the behavior of the blood glucose level.

1. A pulse wave device for quantifying the blood glucose level in a subject and/or for discriminating between different sources of blood glucose level changes, wherein sources of glucose level changes are caused from the type of nutrients, type of sport activities, type of stresses and/or fatigue or a combination thereof, said pulse wave device is adapted to be applied on a pulse-taking location on the body of said subject; said pulse wave device comprising:
a sensor module for collecting information data from a pulse wave, a memory module for storing the pulse wave information data on the pulse wave device, a display module for displaying the results of the blood glucose level and/or the discrimination between said different sources of blood glucose level changes and a processor module comprising:
means for extracting and selecting from each single pulse wave and from its first and second derivation a first set of features determined by measuring the entire pulse wave timeline, or by identifying a set of pulse wave points selected from: the systolic peak, diastolic peak, dicrotic notch, the first and last points corresponding to the half-height of the systolic peak, and the starting and ending points of said single pulse wave providing information data consisting in the time, amplitude or area of the pulse wave, ratios in said first set of features, heart rate and breathing rate of said subject;
wherein, said processor module is configured to perform a statistical analysis on the collected information data from the pulse wave and/or on said first set of features obtained from at least two single pulse waves to arrive at a second set of features providing additional information data consisting in the mean, variation around the mean, randomness and/or time series analysis between said first set of features of the at least two single pulse waves; and wherein, said processor module further comprises means configured to combine said first and second set of features and means to analyze and display the results of the blood glucose level and/or the discrimination between said different sources of blood glucose level changes of said subject.

2. The pulse wave device according to claim 1, wherein the pulse wave device is further adapted to identify diabetic or pre-diabetic subjects from healthy subjects and wherein diabetes or pre-diabetes comprises Type I diabetes, Type II diabetes, hyperglycemia impaired fasting glucose and impaired. glucose tolerance.

3. The pulse wave device according to claim 1, wherein time series analysis are performed by ANN, RNN, DL or CNN techniques.

4. The pulse wave device according to claim 1, wherein said pulse wave device is adapted for personal health care diagnosis.

5. The pulse wave device according to claim 1, wherein said pulse wave device further comprises a warning unit capable of alerting the subject when a certain level of blood glucose has been reached.

6. The pulse wave device according to claim 1, wherein said sensor module for collecting information data from said single pulse wave are selected among pulse taking sensors, photo or video imaging, smart phone camera, optical emitters based on LEDS, pulse oximeters, or a combination thereof.

7. The pulse wave device according to claim 1, wherein said pulse wave device is configured to provide an output without filtering the output and distorting the pulse wave shape.

8. (canceled)

9. The pulse wave device according to claim 1, wherein ratios in said first set of features comprise:
a ratio of an amplitude of a systolic peak and an amplitude of a diastolic peak;—A ratio of the amplitude of the systolic peak and an amplitude of a dicrotic notch;
a ratio of the amplitude of the dicrotic notch and the amplitude of the diastolic peak;—A ratio of a time value of the systolic peak and a time value of the diastolic peak;
—a ratio of the time value of the systolic peak and a time value of the dicrotic notch;
a ratio of the time value of the dicrotic notch and the time value of the diastolic peak;—A time difference between the time value of the systolic peak and the time value of the diastolic peak;
a time difference between the time value of the systolic peak and the time value of the diastolic notch;
a time difference between the time value of the dicrotic notch and the time value of the diastolic peak;
a local cardiac output corresponding to a ratio of an area under the curve to a time difference between a starting time and an ending time;
a ratio of the area under the curve between the starting point and the systolic peak to the amplitude of the systolic peak;
a local systolic cardiac output corresponding to a ratio of an area under the curve between the starting point and the dicrotic notch to the time value of the dicrotic notch;
a ratio of an area under the curve between the starting point and the dicrotic notch to the amplitude of the systolic peak;
a local diastolic cardiac output corresponding to a ratio of an area under the curve between the dicrotic notch and the ending point to the time difference between the time value of the dicrotic notch and the time value of the ending point;
a ratio of an area under the curve between the dicrotic notch and the ending point to the amplitude of the diastolic peak;
a pulse width at ten, thirty, fifty, seventy, or ninety percent corresponding to a time difference between the first and the last points corresponding ten, thirty, fifty, seventy, or ninety percent of the systolic peak, respectively;
a time difference between the first point corresponding to ten, thirty, fifty, seventy, or ninety percent of the systolic peak and the systolic time;
a time difference between the systolic peak and the last point corresponding to ten, thirty, fifty, seventy, or ninety percent of the systolic peak;
a pulse interval corresponding to the time difference between the ending and starting time;
a slope of the systolic peak corresponding to the ratio of the amplitude of the systolic peak by the time value of the systolic peak;
a slope of the diastolic peak corresponding to the ratio of the amplitude of the diastolic peak by the time difference between the ending point and the diastolic peak;
a diastolic decay corresponding to a logarithm of the slope of the diastolic peak;—An inflection point area ratio corresponding to the ratio of the area under the curve between the dicrotic notch and the ending point divided by the area under the curve between the starting point and the dicrotic notch;
an augmentation index, corresponding to the ratio of the amplitude of the systolic peak divided by the amplitude of the diastolic peak;
the ratio of the local diastolic cardiac output by the local systolic cardiac output, or the inverses thereof;
a pulse mean corresponding to the mean of the pulse curve;
a pulse standard deviation corresponding to the standard deviation of the pulse curve;
a pulse median corresponding to the median of the pulse curve;
a ratio of the local systolic cardiac output and the local diastolic cardiac output;
a ratio of the amplitude of the systolic peak minus the amplitude of the dicrotic notch divided by the amplitude of the diastolic peak minus the amplitude of the dicrotic notch;
a ratio of the area under the curve between the systolic peak and the dicrotic notch to the time difference between the time of the systolic peak and the time of the dicrotic notch;
a ratio of the area under the curve between the systolic peak and the dicrotic notch to the amplitude of the systolic peak.

10. The pulse wave device according to claim 1, wherein said variation around the mean in said second set of features consists of skewness, variance, standard deviation and power spectrum.

11. The pulse wave device according to claim 1, wherein said randomness in said second set of features consists of entropy.

12. The pulse wave device according to claim 1, wherein the processor module is configured to calculate a pre-selected combination of said first and second set of features after a preprocessing step involving the selection of convenient pulse waves and then to apply it to a model programmed in said processor module to determine the blood glucose level and to discriminate between different sources of blood glucose level changes.

13. The pulse wave device according to claim 1, wherein the processor module is configured to select an optimal sub-set of features resulting from the combination of said first and said second set of features through modelling as a sparse regularized optimization and applying greedy mathematical algorithms in order to discriminate at least one of said blood glucose level changes selected among the type of nutrients, type of sport activities, type of stresses and fatigue or a combination thereof.

14. A statistical and analytic non-invasive method for interpreting a set of pulse wave recordation of a subject for quantifying the blood glucose level and/or discriminating between different sources of blood glucose level changes caused from the type of nutrients, type of sport activities, type of stresses and fatigue or a combination thereof, said method comprising the steps of:
extracting and selecting from said set of pulse wave recordation each single pulse wave and its first and second derivation so as to obtain a first set of features determined by measuring the entire pulse wave timeline, or by identifying a set of pulse wave points selected from: the systolic peak, diastolic peak, dicrotic notch, the first and last points corresponding to the half-height of the systolic peak, and the starting and ending points of said single pulse wave providing information data consisting in the time, amplitude or area of the pulse wave, ratios in said first set of features, heart rate and breathing rate of said subject; wherein, the method is performing a statistical analysis on the collected information data from the pulse wave and/or on said first set of features obtained from at least two single pulse waves to arrive at a second set of features providing additional information data consisting in the mean, variation around the mean, randomness and/or time series analysis between said first set of features of the at least two single pulse waves; and wherein the method is combining said first and second set of features and applying means configured in a software to analyze, determine and display the results of the blood glucose level and/or of the discrimination between different sources of blood glucose level changes of said subject.

15. The statistical and analytic non-invasive method according to claim 14, wherein time series analysis are performed by ANN, RNN, DL or CNN techniques.

16. (canceled)

17. The statistical and analytic method according to claim 14, wherein ratios in said first set of features comprise:
a ratio of an amplitude of a systolic peak and an amplitude of a diastolic peak;
a ratio of the amplitude of the systolic peak and an amplitude of a dicrotic notch;
a ratio of the amplitude of the dicrotic notch and the amplitude of the diastolic peak;
a ratio of a time value of the systolic peak and a time value of the diastolic peak;
a ratio of the time value of the systolic peak and a time value of the dicrotic notch;
a ratio of the time value of the dicrotic notch and the time value of the diastolic peak;
a time difference between the time value of the systolic peak and the time value of the diastolic peak;
a time difference between the time value of the systolic peak and the time value of the dicrotic notch;
a time difference between the time value of the dicrotic notch and the time value of the diastolic peak;
a local cardiac output corresponding to a ratio of an area under the curve to a time difference between a starting time and an ending time;
a ratio of the area under the curve between the starting point and the systolic peak to the amplitude of the systolic peak;
a local systolic cardiac output corresponding to a ratio of an area under the curve between the starting point and the dicrotic notch to the time value of the dicrotic notch;
a ratio of an area under the curve between the starting point and the dicrotic notch to the amplitude of the systolic peak;
a local diastolic cardiac output corresponding to a ratio of an area under the curve between the dicrotic notch and the ending point to the time difference between the time value of the dicrotic notch and the time value of the ending point;
a ratio of an area under the curve between the dicrotic notch and the ending point to the amplitude of the diastolic peak;
a pulse width at ten, thirty, fifty, seventy, or ninety percent corresponding to a time difference between the first and the last points corresponding ten, thirty, fifty, seventy, or ninety percent of the systolic peak, respectively;
a time difference between the first point corresponding to ten, thirty, fifty, seventy, or ninety percent of the systolic peak and the systolic time;
a time difference between the systolic peak and the last point corresponding to ten, thirty, fifty, seventy, or ninety percent of the systolic peak;
a pulse interval corresponding to the time difference between the ending and starting time;
a slope of the systolic peak corresponding to the ratio of the amplitude of the systolic peak by the time value of the systolic peak;
a slope of the diastolic peak corresponding to the ratio of the amplitude of the diastolic peak by the time difference between the g point and the diastolic peak;
a diastolic decay corresponding to a logarithm of the slope of the diastolic peak;
an inflection point area ratio corresponding to the ratio of the area under the curve between the dicrotic notch and the ending point divided by the area under the curve between the starting point and the dicrotic notch;
an augmentation index, corresponding to the ratio of the amplitude of the systolic peak divided by the amplitude: of the diastolic peak;
the ratio of the local diastolic cardiac output by the local systolic cardia, output, or the inverses thereof;
a pulse mean corresponding to the mean of the pulse curve;
a pulse standard deviation corresponding to the standard deviation of the pulse curve;
a pulse median corresponding to the median of the pulse curve;
a ratio of the local systolic cardiac output and the local diastolic cardiac output;
a ratio of the amplitude of the systolic peak minus the amplitude of the dicrotic notch divided by the amplitude of the diastolic peak minus the amplitude of the dicrotic notch;
a ratio of the area under the curve between the systolic peak and the dicrotic notch to the time difference between the time of the systolic peak and the time of the dicrotic notch;
a ratio of the area under the curve between the systolic peak and the dicrotic notch to the amplitude of the systolic peak.

18. The statistical and analytic method according to claim 14, wherein said variation around the mean in said second set of features consists of skewness, variance and standard deviation.

19. The statistical and analytic method according to claim 14, wherein said randomness in said second set of features consists of entropy.

20. The statistical and analytic method according to claim 14, wherein the software is configured to calculate a pre-selected combination of said first and second set of features after a preprocessing step involving the selection of convenient pulse waves and then apply it to a model programmed in said software to determine the blood glucose level and to discriminate between different sources of blood glucose level changes.

21. The statistical and analytic method according to claim 14, wherein the software is configured to select an optimal sub-set of features resulting from the combination of said first and said second set of features through modelling as a sparse regularized optimization and applying greedy mathematical algorithms in order to discriminate at least one of said blood glucose level changes caused from the type of nutrients, type of sport activities, type of stresses and fatigue or a combination thereof.
